(19) United States
(12) Patent Application Publication
(10) Pub. No.: US 2024/0010605 A1
(43) Pub. Date: Jan. 11, 2024

(54) COMPOUND, PREPARATION METHOD THEREFOR AND APPLICATION OF COMPOUND IN PREPARATION OF BICYCLOPYRONE INTERMEDIATE

(71) Applicant: SHANDONG WEIFANG RAINBOW CHEMICAL CO., LTD, Weifang (CN)

(72) Inventors: Zhiqing LI, Weifang (CN); Fenghua Gong, Weifang (CN); Haichao Zhang, Weifang (CN); Hongquan Zhang, Weifang (CN); Xiaoyang Qiu, Weifang (CN)

(21) Appl. No.: 18/453,549

(22) Filed: Aug. 22, 2023

Related U.S. Application Data
(63) Continuation of application No. PCT/CN2023/098992, filed on Jun. 7, 2023.

(30) Foreign Application Priority Data
Jun. 8, 2022 (CN) 202210637542.4

(51) Int. Cl. C07C 67/42; C07C 69/738; C07D 309/32

(52) U.S. Cl. CPC C07C 67/42; C07C 69/738; C07D 309/32

(57) ABSTRACT

Through an intermediate I (a compound having the structural formula as shown in the formula Ia and/or the formula Ib), or a pharmaceutically acceptable salt thereof, or a solvate thereof, and a tautomer Ic of Ib, a bicyclopyrone intermediate II with a high yield can be prepared. Two compounds are docked first under the action of a base to produce an intermediate I, and then the intermediate I is subjected to intramolecular ring closure by an ammonium salt, which can increase the yield of the bicyclopyrone intermediate (II), reduce side reactions, and reduce problems that a reaction of raw materials is easily incomplete due to intramolecular ring closure directly through an ammonium salt. A one-pot method includes producing an intermediate I under the action of a base and then performing a ring-closure reaction to produce a bicyclopyrone intermediate (II) that reduces side reactions, and further increases the yield.

### CROSS-REFERENCE

The present application claims the right of priority for Chinese patent application No. 202210637542.4, filed to the China National Intellectual Property Administration on Jun. 8, 2022 and entitled “Compound, Preparation Method Therefor and Application of Compound in Preparation of Bicyclopyrone Intermediate”, which is incorporated herein in its entirety by reference.

### TECHNICAL FIELD

The present application relates to the technical field of pesticides, in particular to a compound, a preparation method therefor and an application of the compound in the preparation of a bicyclopyrone intermediate.

### BACKGROUND ART

Bicyclopyrone has been first marketed in 2015, and has been registered and marketed in United States, Canada, Argentina, Uruguay, Australia and other countries. As a kind of 4-hydroxyphenylpyruvate dioxygenase (HPPD) inhibitors, bicyclopyrone destroys chlorophyll in plants, and may be combined with various herbicides such as mesotrione, isoxaflutole, topramezone, tembotrione, and pyrasulfatole. Such selected herbicides have good activity to broadleaf weeds, and perennial and annual weeds, and may be used for corn fields, wheat fields, barley fields, sugarcane fields and other crop fields. Bicyclopyrone has the following structure:

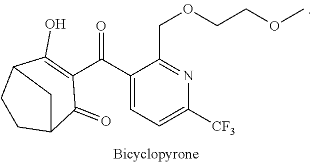 [SMILES: COCCOCc1nc(C(F)(F)F)ccc1C(=O)C1=C(O)C2CCC(C2)C1=O]

A current typical process of bicyclopyrone (CN1824662B) is reported as follows:

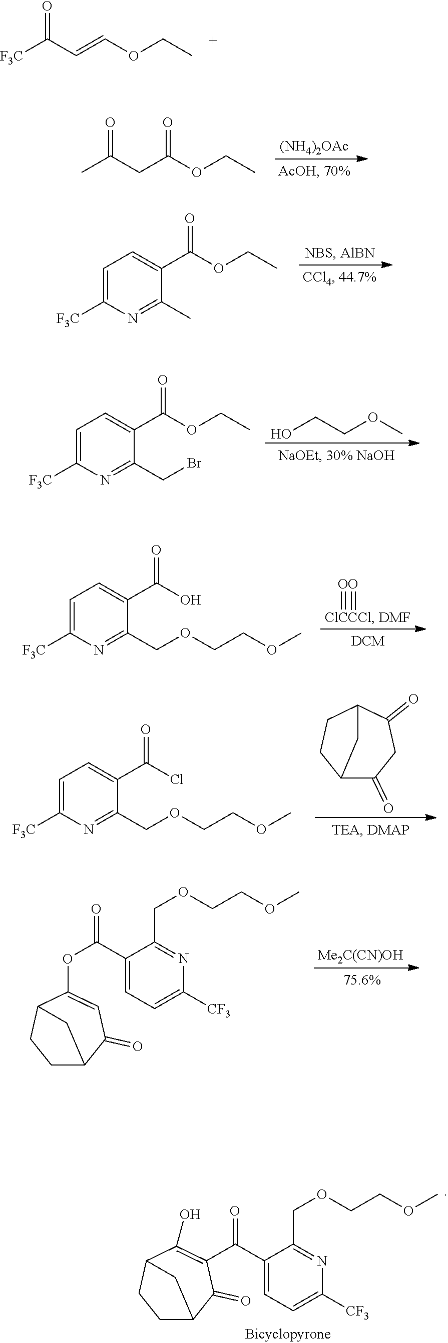

An experiment shows that in the first step, a reaction is difficult in ring closure; in the second step, the bromine substitution selectivity of methyl is not good, and the yield is only 44.7% (see CN 1824662 B); and in the third step, an etherification reaction is prone to producing lactone impurities, and there is no yield data provided in literatures. In the above reaction route, NBS is N-bromosuccinimide; AIBN is azobisisobutyronitrile; DMF is N, N-dimethylformamide; DCM is dichloromethane; TEA is triethylamine; and DMAP is 4-dimethylaminopyridine.

It is reported in the literatures (A convenient and effective method for synthesizing β-amino-α, β-unsaturated esters and ketones, Synthetic Communications, 2004, 34 (5), 909-916, and Synthesis of functionalized pyridinium salts bearing a free amino group, ARKIVOC, 2014, 2014 (3), 154-169) that ethyl (Z)-3-aminobut-2-enoate with a quantitative yield is obtained from ethyl acetoacetate in methanol with ammonium carbamate, and is further condensed with vinyl ether to obtain ethyl 2-methyl-6-(trifluoromethyl) nicotinate (see patent WO2006059103A2), similar to the above route. The reaction route is as follows:

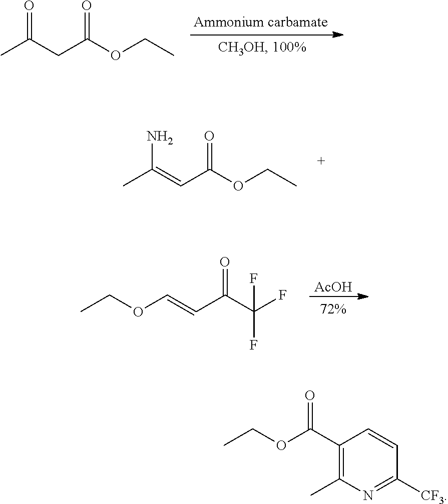

The patent WO2006059103A2 has reported a method for synthesizing ethyl 2-methyl-6-(trifluoromethyl) nicotinate. The method is widely used by most manufacturers at present. The experiment shows that the reaction is incomplete in conversion, there is no effect if a reaction time is prolonged, and main by-products are two enamines. The reaction route is as follows:

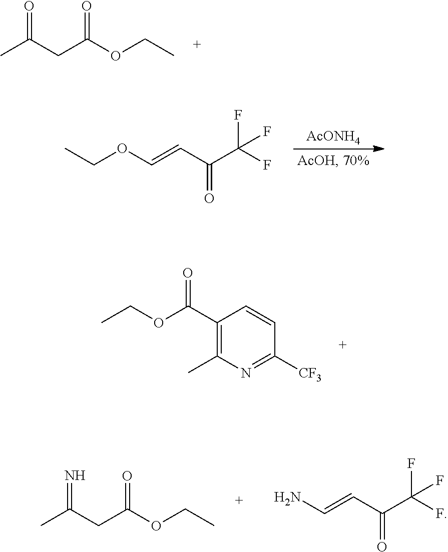

The patent WO2006059103A2 has reported a preparation method of ethyl 2-(chloromethyl)-6-(trifluoromethyl) nicotinate from ethyl 4-chloro-3-oxobutanoate and 4-ethoxy-1,1,1-trifluorobut-3-en-2-one with a one-pot method in the presence of acetic acid and ammonium acetate. The experiment shows that the product of the method is messy, and is low in a yield and difficult in purification; whether the temperature is increased or the time is prolonged, a residual intermediate of the enamine still presents, and can only be separated by column chromatography; and the product cannot be applied to production. The reaction route is as follows:

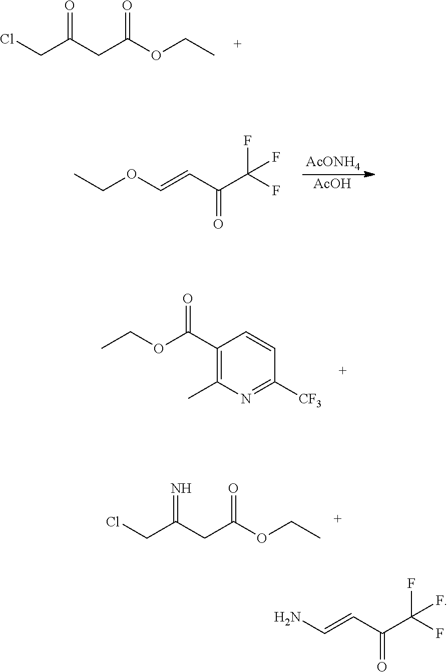

Syngenta's patent WO2004078729A1 has reported that ethyl 4-chloro-3-oxobutanoate and 2-methoxyethanol are docked in tetrahydrofuran (THF) under the action of NaH to obtain an etherified product; and then ammoniation and ring closure are performed to prepare ethyl 2-((2-methoxyethoxy)methyl)-6-(trifluoromethyl) nicotinate. According to a record, a linear yield is expected to be 36%. In the reaction route, TFA refers to trifluoroacetic acid. There are many problems in the route, for example, sodium hydride is used in the reaction in the first step. Excessive use of sodium hydride makes it very difficult to separate and purify products with two or more intermolecular macromolecules formed between or with ethyl 4-chloro-3-oxobutanoate; anhydrous tetrahydrofuran needs to be used, and the reaction conditions are strict; and introducing ammonia gas in the second step is a dangerous reaction, although it is controllable. The reaction route is as follows:

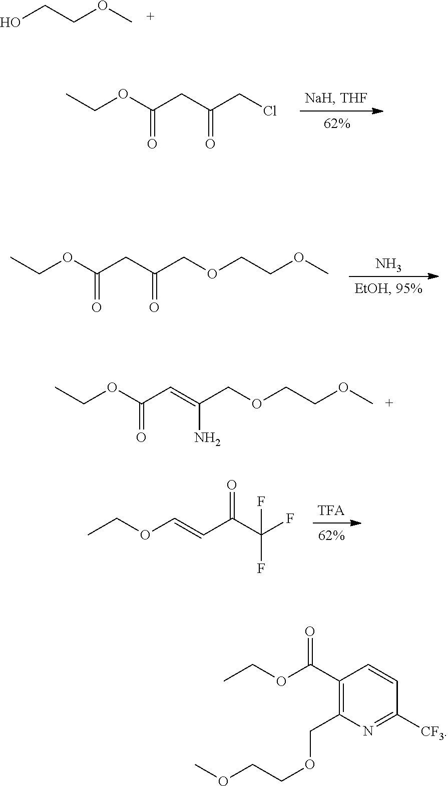

In the third step, the reaction has a low ring-closure yield, the main reason for which is that the reaction of the reactant enamine to vinyl ether are more in active functional groups and side reactions. As the reaction progresses, the produced water hydrolyzes enamine back to ketone, which cannot be further converted to a product, resulting in incomplete reaction of raw materials. The side reactions are as follows:

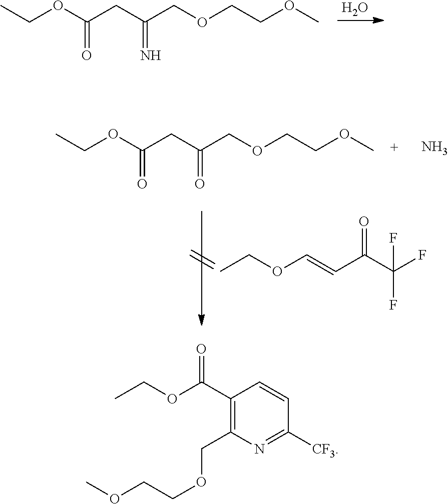

The patent WO2016102347A1 gives a synthesis method introducing a side chain. Two starting materials in the method are not easy to obtain, relatively high in the cost which is further increased with the use of magnesium ethoxide, N, N-carbonyldiimidazole (CDI) and anhydrous tetrahydrofuran, poor in the atomic economy, and high in the purification cost, and thus having no industrial application value. Similarly, the method of the patent WO2005026149 cannot be applied to production. The reaction routes are as follows:

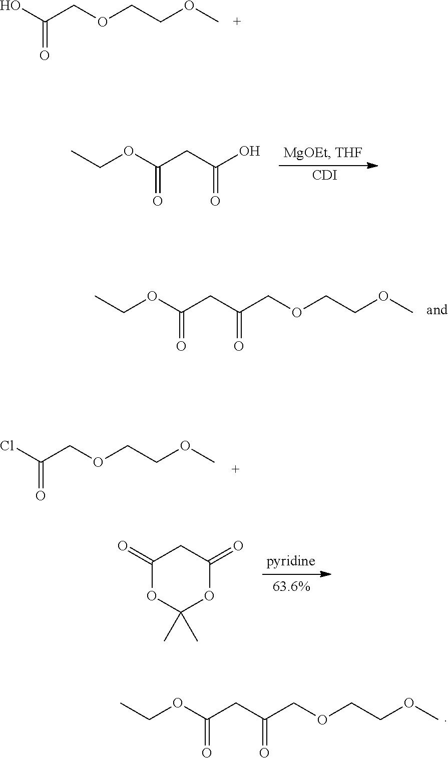

In 2015, the patent EP2821399A1 from Lonza Ltd has reported another synthesis method, in which a linear yield is increased to a certain extent; however, the starting materials are difficult to synthesize, the steps are long, and more wastes are produced, for example, phosphorus-containing wastewater is difficult to treat, expensive catalysts need to be used, trifluoroacetyl chloride is unstable, and the like. The cost is also very high. The reaction route is as follows:

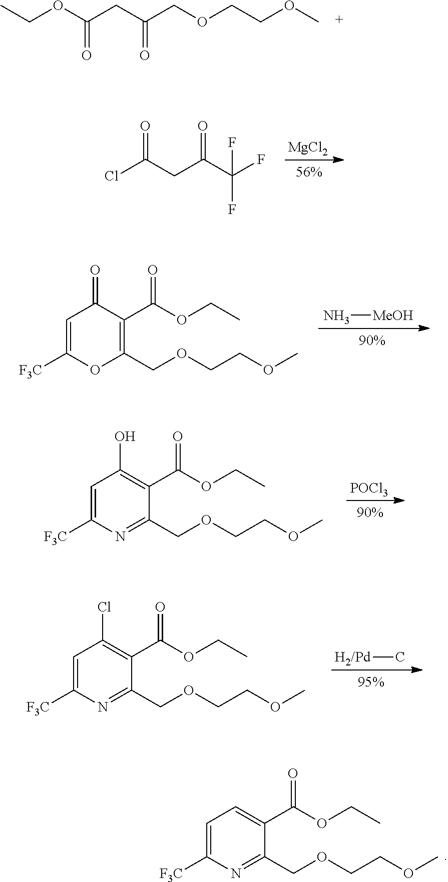

In summary, there is no process route that is readily available in raw materials, safe and reliable, and suitable for industrial amplification.

### SUMMARY OF THE INVENTION

### Invention Objective

In order to overcome the above shortcomings, the objective of the present application is to provide a compound, a preparation method therefor, and an application of the compound in the preparation of a bicyclopyrone intermediate. The bicyclopyrone intermediate includes an intermediate I (compound(s) shown in the formula (Ia) and/or the formula (Ib)) and an intermediate II (compound(s) shown in the formula IIA, the formula IIB and the formula IIC, belonging to nicotinic acid fragments).

In the present application, two fragments for preparing nicotinic acid are docked first under the action of a base to produce an intermediate I, and then the intermediate I is subjected to intramolecular ring closure by an ammonium salt, which can remarkably increase the yield of the bicyclopyrone intermediate (II), reduce side reactions, and overcome the defects in the prior art (for example, methods reported in WO2006059103A2 and WO2004078729A1) that a reaction of raw materials is easily incomplete due to intramolecular ring closure directly by an ammonium salt. In the present application, the one-pot method of producing an intermediate I under the action of a base and then performing a ring-closure reaction to produce a bicyclopyrone intermediate (II), can reduce side reactions, and can further increase the yield.

### Solution

In order to achieve the objective of the present application, the technical solution used by the present application is as follows:

In the first aspect, the present application provides a compound (named intermediate I), having a structural formula as shown in the formula Ia and/or the formula Ib, or a pharmaceutically acceptable salt thereof, or a solvate thereof, or a tautomer Ic of Ib,

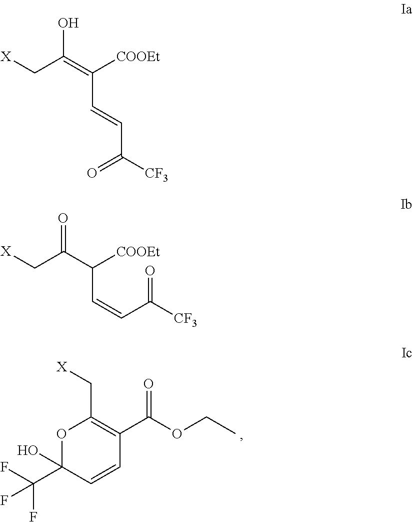

wherein X is —O—R¹—O—R², —H, —Cl or —Br; and if X is —O—R¹—O—R², R¹ is selected from C₁-C₄ alkylene groups, and R² is selected from C₁-C₄ alkyl groups.

In a further research, it is found that the structure of the formula Ib is a pair of keto racemic isomers in a base environment of the reaction, but the pair of keto racemic isomers exist in a form of hemiketal isomers (such as the structure as shown in the formula Ic) after quenching with an acid; and it is then found that the structure of hemiketal is more stable, and also mainly presents in a form of hemiketal in a dry product.

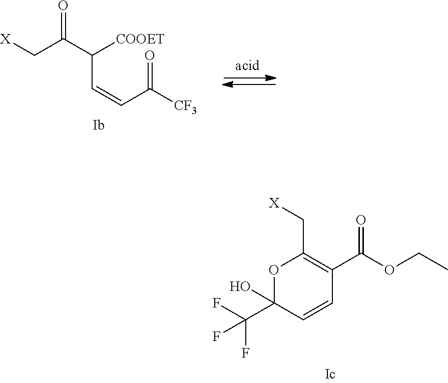

Further, if X is hydrogen, the compound is a compound having a structural formula as shown in the formula (Ia-1) or the formula (Ib-1), and a tautomer of the formula (Ib-1) is shown in the formula (Ic-1).

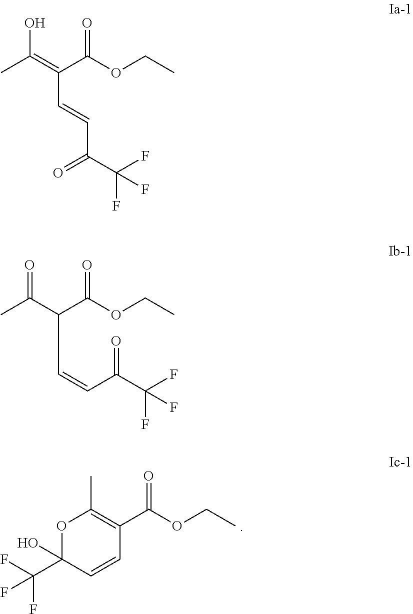

Or, X is —Cl or —Br; optionally, if X is —Cl, the compound is a compound having a structural formula as shown in the formula (Ia-2) or the formula (Ib-2), and a tautomer of the formula (Ib-2) is shown in the formula (Ic-2).

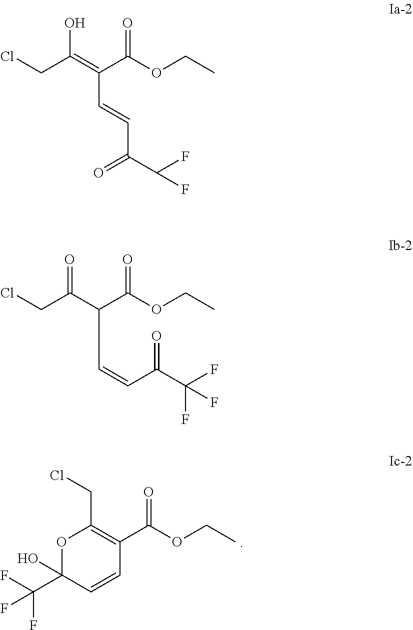 [SMILES: CCOC(=O)C(/C=C/C(=O)C(F)F)=C(\O)CCl ; CCOC(=O)C(/C=C\C(=O)C(F)(F)F)C(=O)CCl ; CCOC(=O)C1=C(CCl)OC(O)(C(F)(F)F)C=C1]

Or, X is —O—R¹—O—R², R¹ is selected from C₁-C₄ alkylene groups, and R² is selected from C₁-C₂ alkyl groups. Optionally, R¹ is selected from C₁-C₃ alkylene groups, and R² is selected from C₁-C₂ alkyl groups. Optionally, R¹ is selected from C₂-C₃ alkylene groups, and R² is selected from C₁-C₂ alkyl groups. Preferably, if X is —O(CH₂)₂OCH₃, the compound is a compound having a structural formula as shown in the formula Ib-3, or a pharmaceutically acceptable salt thereof, or a tautomer as shown in the formula Ic-3,

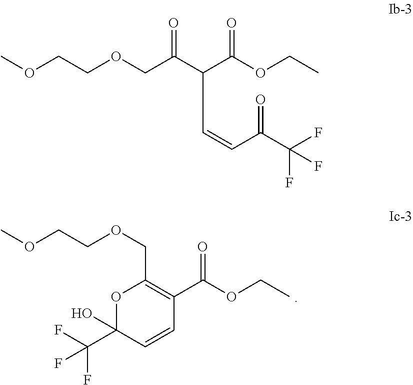 [SMILES: CCOC(=O)C(/C=C\C(=O)C(F)(F)F)C(=O)COCCOC ; CCOC(=O)C1=C(COCCOC)OC(O)(C(F)(F)F)C=C1]

In the second aspect, the present application provides a preparation method for the compound in the first aspect, comprising the following step:
in the presence of a base, making a compound as shown in the formula III and/or an enol tautomer thereof subjected to a substitution reaction with a compound as shown in the formula IV, to obtain a compound as shown in the formula Ia and/or Ib and/or Ic,

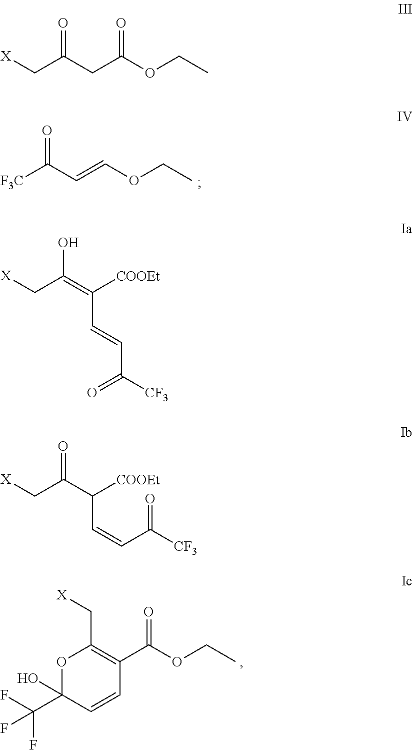

wherein X is —O—R¹—O—R², —H, —Cl or —Br; and if X is —O—R¹—O—R, R¹ is selected from C₁-C₄ alkylene groups, and R² is selected from C₁-C₄ alkyl groups; and in the formulas Ia, Ib, Ic, II and III, X is the same.

In the third aspect, the present application provides a preparation method for a bicyclopyrone intermediate, including the following steps:
1) in the presence of a base, making a compound as shown in the formula III and/or an enol tautomer thereof subjected to a substitution reaction with a compound as shown in the formula IV,

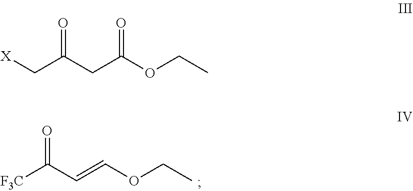

and
2) in the presence of an ammonium salt and/or ammonia, making a product of the substitution reaction in step 1) subjected to a ring-closure reaction to obtain a compound as shown in the formula II, wherein in the formula II and the formula III, X is the same,

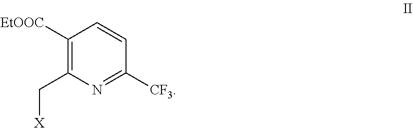

In the preparation method in the second aspect or the third aspect, in the substitution reaction, the base is one or more selected from the group consisting of an organic base, an inorganic base, sodium hydride or metal sodium; optionally, the organic base includes one or more of sodium alkoxide and potassium alkoxide; optionally, the organic base includes one or more of sodium methoxide, sodium ethoxide, potassium tert-butoxide, potassium methoxide, potassium ethoxide, sodium hexamethyldisilazane and lithium hexamethyldisilazane; optionally, the inorganic base includes one or more of sodium hydroxide, potassium hydroxide, sodium carbonate, potassium carbonate, sodium phosphate, potassium phosphate and sodium amide; and optionally, the base is one or more selected from the group consisting of sodium ethoxide, sodium hydroxide and sodium carbonate.

In the preparation method in the second aspect or the third aspect, the substitution reaction is performed in an organic solvent; optionally, the organic solvent includes one or more of organic alcohol, toluene, tetrahydrofuran, dimethyl sulfoxide (DMSO), N, N-dimethylformamide (DMF), and 1, 4-dioxane; optionally, the organic solvent includes one or more of methanol, ethanol and toluene; and optionally, the organic solvent includes toluene and/or ethanol.

In the preparation method in the second aspect or the third aspect, in the substitution reaction, a molar ratio of the compound as shown in the formula IV, the compound as shown in the formula III and/or an enol tautomer thereof and the base is 1:0.8-1.5:0.05-1.5, optionally, 1:0.8-1.2:0.5-1.3, optionally, 1:0.9-1.1:1-1.3, and optionally, 1:1: 1-1.3, preferably, 1:1: 1-1.2.

In the preparation method in the second aspect or the third aspect, in the substitution reaction, a reaction temperature is −15° C. to 30° C.; and optionally, 0° C.-25° C., preferably, 0° C.-10° C.

Further, the base is slowly added, and added dropwise in the case of being a solution.

A reaction route of the intermediate I (i.e. the compound in the first aspect) of the present application may be as follows:

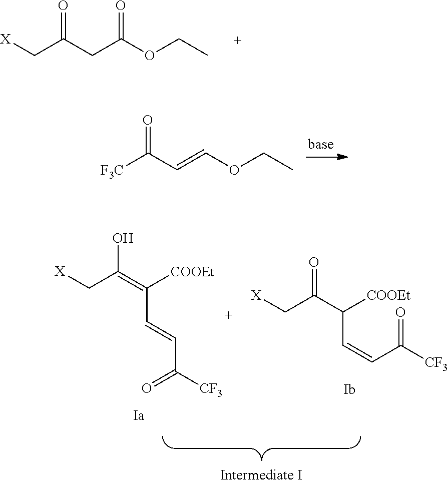

If X is different substituents, a proportion of the tautomers (Ia) and (Ib), which are the enol compound Ia and the keto compound Ib respectively, in the intermediate I can also be different. In addition, (Ib) is prone to being converted to (Ic) by intramolecular ring closure after quenching with an acid after reaction. For convenience in description, in the present application, Ia and Ib before quenching represent reaction products. It should be noted that Ib mainly exists in a form of Ic after quenching with an acid.

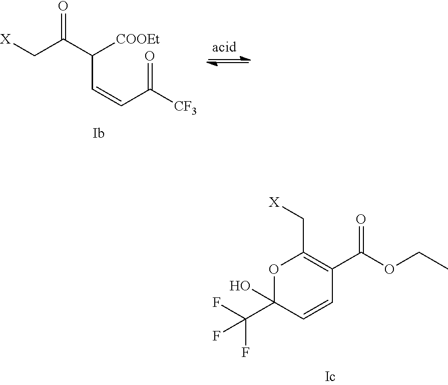

For example, if X is hydrogen, the produced reaction product includes the tautomers as shown in the formulas (Ia-1) and (Ib-1). The reaction route is named route A-1. The reaction route A-1 with sodium ethoxide as the base is as follows:

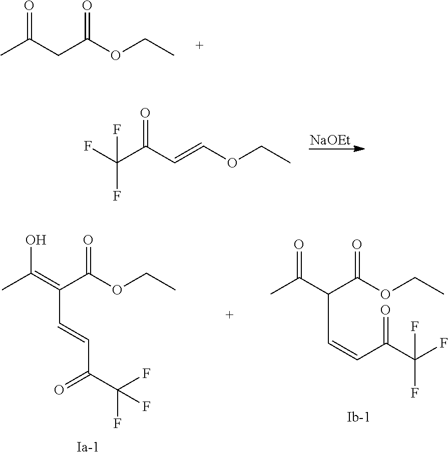

In the reaction route A-1, the intermediate I (having the structural formulas (Ia-1) and (Ib-1)) is obtained by a stepwise method, and then the intermediate IIA is synthesized, which can remarkably reduce side reactions, improve the selectivity of the reaction, achieve a ring-closure reaction under a gentle condition, and increase the yield from 67% to 77.4% (refer to Example 1).

As an alternative, if X is —Cl, the produced reaction product includes the tautomers as shown in the formulas (Ia-2) and (Ib-2). A produced specific impurity includes at least one of an impurity compound A, an impurity compound B, an impurity compound C and an impurity compound D, wherein the specific impurity includes 2-15% of the impurity compound B and 1-5% of the impurity compound C. The reaction route is named route B-1. The reaction route B-1 with sodium ethoxide as the base is as follows:

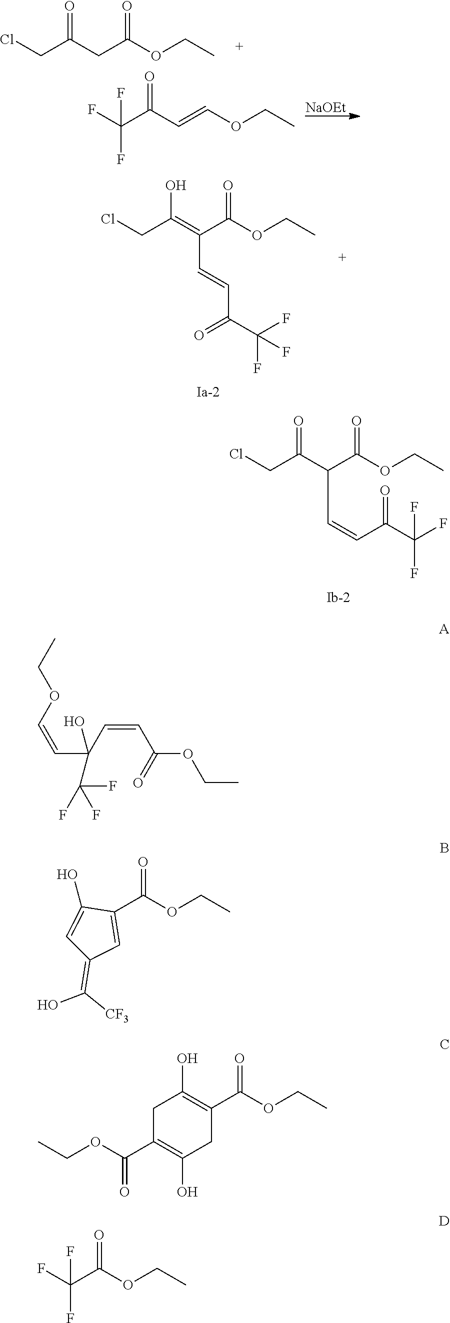

In the reaction route B-1, in the process of obtaining the intermediate I (having the structural formulas (Ia-2) and (Ib-2)) by the stepwise method, under the action of the base, a plurality of sensitive groups present in ethyl 4-chloroacetoacetate, so that the reaction is very complex; and there are still four main by-products: impurity compounds A, B, C and D besides a main product I (a pair of tautomers, i.e. the compounds having the structural formula as shown in the formulas (Ia-2) and (Ib-2)). The intermediate I is further subjected to ring closure to obtain an intermediate II with a total yield of 58% (refer to Example 2), which is also superior to that in the method reported in WO2006059103A2.

Optionally, if IV was added dropwise with the compound as shown in the formula III and the base as substrates, the impurity in the product of the substitution reaction at least includes an impurity compound A, an impurity compound B, an impurity compound C and an impurity compound D.

Optionally, if III and IV are used as substrates, the base is slowly added. When a dropping speed of the base is controlled, the product of the substitution reaction at least includes an impurity compound B, an impurity compound C and an impurity compound D. If the base is a base solution, the base is added dropwise, and the dropping speed is uniformly and completely dropping within 1-3 h, preferably within 1.5-2 h.

As an alternative, X is —O—R¹—O—R², R¹ is selected from C₁-C₄ alkylene groups, and R² is selected from C₁-C₂ alkyl groups. Optionally, R¹ is selected from C₁-C₃ alkylene groups, and R² is selected from C₁-C₂ alkyl groups. Optionally, R¹ is selected from C₂-C₃ alkylene groups, and R² is selected from C₁-C₂ alkyl groups. Preferably, if X is —O(CH₂)₂OCH₃, a produced reaction product is mainly a compound having the structural formula as shown in the formula (Ib-3) (which is prone to being converted to (Ic-3) by intramolecular ring closure under a condition of acid addition); and the produced specific impurity includes at least one of an impurity compound C, an impurity compound D and an impurity compound E. The reaction route is named route C-1. The reaction route C-1 with sodium ethoxide or sodium carbonate as the base is as follows:

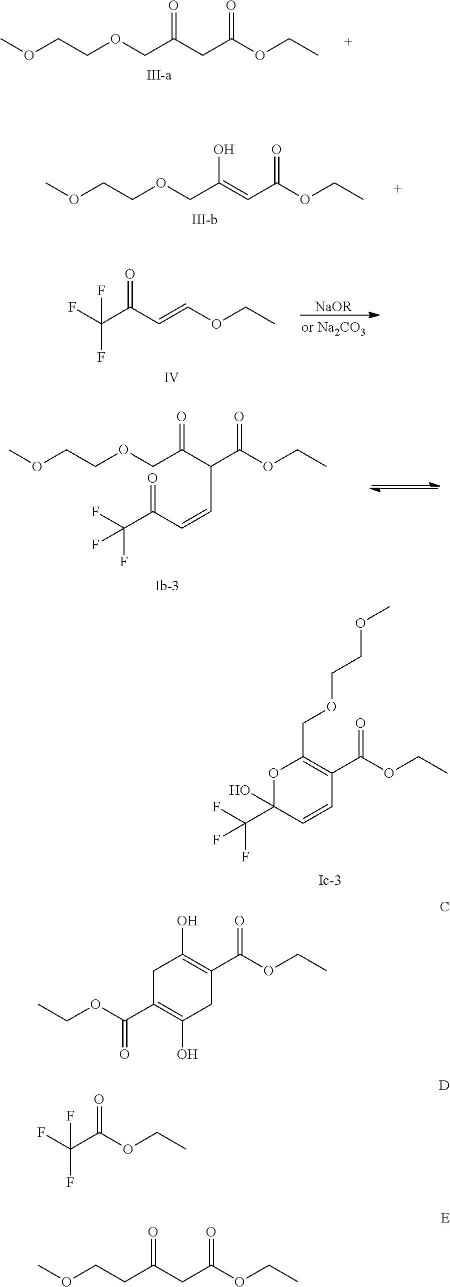

For the intermediate I (Ia and/or Ib and/or Ic), if X is —H, —Cl and —O(CH₂)₂OCH₃, with the increase of a substituent, a content of the intermediate I in an enol form is decreased sequentially, while that a content of the intermediate I in a keto form is in turn increased sequentially; after quenching, if X is —H and —Cl, the isomerization of the ketone form to hemiketal is not obvious; however, if X is —O(CH₂)₂OCH₃, the ketone form is basically completely isomerized into hemiketal.

Generally, the intermediate I (Ia and/or Ib and its tautomer Ic) is conducive to formation of a kinetic product at a low temperature; while in the case of a high temperature, it is conducive to formation of a thermodynamic product. The inventors of the present application find through a research that the intermediate I having a cis structure (keto Ib and a tautomer hemiketal Ic thereof) is conducive to the subsequent ring-closure reaction of the intermediate II; while a trans structure (enol Ia) is difficult in ring closure, and requires a higher temperature. For example, in the route C-1, if the intermediate I (as shown in the formula (Ib-3) and/or the formula (Ic-3)) is changed into a trans Ia-3 structure, due to the steric hindrance of the side chain and high activation energy of double bond inversion required by ring closure, ring closure cannot be achieved smoothly in a low temperature range.

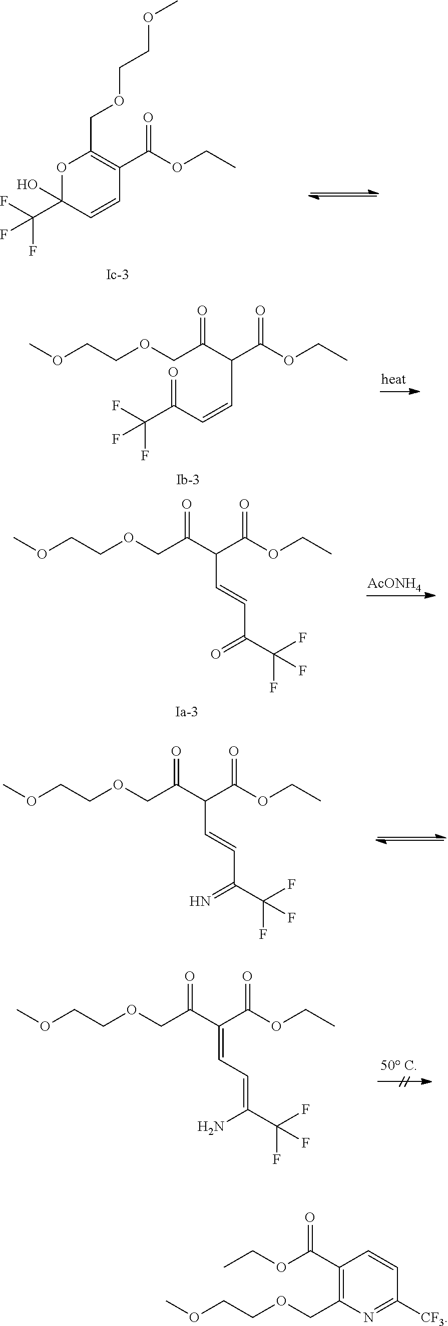

The reaction route C-1 is different from the reaction routes A-1 and B-1 in that the intermediate I obtained by the stepwise method generally only has a keto cis product having the structural formula (Ib-3); during the acid quenching reaction Ib-3 is easily isomerized into a hemiketal product having the structural formula Ic-3 (in a further research, it is found that Ic-3 is still conducive to a subsequent reaction after conversion); however, at elevated temperatures or under more acidic conditions, a small amount of enol trans product Ia-3 may also be produced. Due to less or even no enol trans product Ia-3 produced in the route C-1, the keto cis product (Ib-3) is produced in the one-pot method, and then directly used for subsequent reactions without an acid quenching reaction, which is more conducive to increasing the reaction yield. The hemiketal cis product (Ic-3) obtained from isomerization of (Ib-3) by the acid quenching reaction in the stepwise method is also conducive to subsequent purification of the nicotinic acid. Therefore, the method of the route C-1 is better than those of the routes A-1 and B-1.

In the above reaction routes A-1, B-1, and C-1, the intermediate I (Ia-1 and Ib-1, or Ia-2 and Ib-2, or Ib-3) is prepared first, and then the intermediate II is prepared, which is of great significance to reduce production of by-products, achieve large-scale production and reduce the production cost; and the intermediate II can be obtained with a high conversion rate and a high yield. Among them, the route C-1 is high in a linear yield, few in by-products and more suitable for industrial large-scale production.

Among the above three solutions A-1, B-1 and C-1, the solution C-1 is a preferred route due to few side reactions, for example, avoiding the disadvantages in the route A-1 of poor selectivity of methyl halogenation and the disadvantages in the route B-1 of more side reactions of ethyl 4-chloroacetoacetate.

In the preparation method in the third aspect, the condition for the ring-closure reaction includes: a temperature is 0° C.-80° C., preferably, 45° C.-60° C.; In the preparation method in the third aspect, in the ring-closure reaction, the ammonium salt includes one or more of ammonium chloride, ammonium carbonate, ammonium bicarbonate, ammonium nitrate, ammonium sulfate, ammonium phosphate, and ammonium acetate, preferably, the ammonium salt is ammonium acetate; and the ammonia exists in a form of ammonia gas and/or ammonia water.

In the preparation method in the third aspect, in the ring-closure reaction, a molar ratio of the compound as shown in the formula Ia and/or the formula Ib to the ammonium salt and/or the ammonia is 1: 1-5, optionally, 1: 1-2.5, preferably, 1:1.2-1.5.

In the fourth aspect, the present application provides a composition, including the compound in the first aspect, or a product prepared with the preparation method in the second aspect or the third aspect.

As a possible embodiment, if X is H, the composition includes a compound having the structural formulas (Ia-1) and (Ib-1) with a molar ratio of about 5:2 at room temperature (the compounds as shown in the formula Ia-1 and the formula Ib-1 are a pair of tautomers, which are in a chemical equilibrium in a reaction system and have the molar ratio related to the temperature).

As a possible embodiment, if X is Cl, the composition includes compounds having the structural formulas (Ia-2) and (Ib-2) with a molar ratio of about 1:5 at room temperature (the compounds as shown in the formula Ia-2 and the formula Ib-2 are a pair of tautomers, i.e. a pair of reaction products, which are in a chemical equilibrium in a reaction system and have the molar ratio related to the temperature). Optionally, the composition further includes at least one of an impurity compound A, an impurity compound B, an impurity compound C and an impurity compound D; and optionally, the composition further includes 2-15% of an impurity compound B and 1-5% of an impurity compound C.

As a possible embodiment, if X is —O(CH₂)₂OCH₃, the composition includes a compound having the structural formula as shown in the formula (Ib-3); and optionally, the composition further includes at least one of an impurity compound C, an impurity compound D and an impurity compound E.

In the fifth aspect, the present application provides an application of the compound in the first aspect, the product prepared with the preparation method in the second aspect or the third aspect and the composition in the fourth aspect in the preparation of bicyclopyrone.

In the preparation method in the second aspect or the third aspect, the product of the substitution reaction can be directly used for subsequent reactions, or can be purified (such as vacuum distillation) for subsequent reactions.

Preferably, the present application provides a preparation method for a bicyclopyrone intermediate IIC with a one-pot method (that is, a reaction product is directly used for subsequent reactions), including the following steps:
(1) making a material containing a salt of 2-methoxyethanol reacted with ethyl 4-chloroacetoacetate, to obtain a reaction material containing the formula III-a and/or III-b,

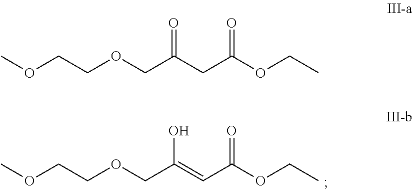

(2) making the reaction material in step (1) subjected to a substitution reaction with a compound as shown in the formula IV under the action of a base, to obtain a reaction material,

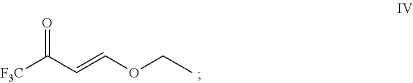

and
(3) adding an ammonium salt and/or ammonia to the reaction material in step (2) to make the reaction material in step (2) subjected to a ring-closure reaction, to obtain a compound as shown in the formula IIC,

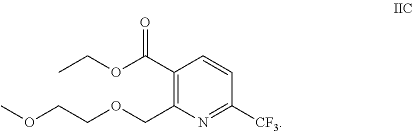 [SMILES: CCOC(=O)c1ccc(C(F)(F)F)nc1COCCOC]

Wherein the compounds as shown in the formula III-a and the formula III-b are a pair of tautomers, and their molar ratio is obtained in the case of achieving equilibrium between the tautomers.

The reaction route in this step is the reaction route C-1 of the one-pot method (with the base being sodium alkoxide or sodium carbonate as an example):

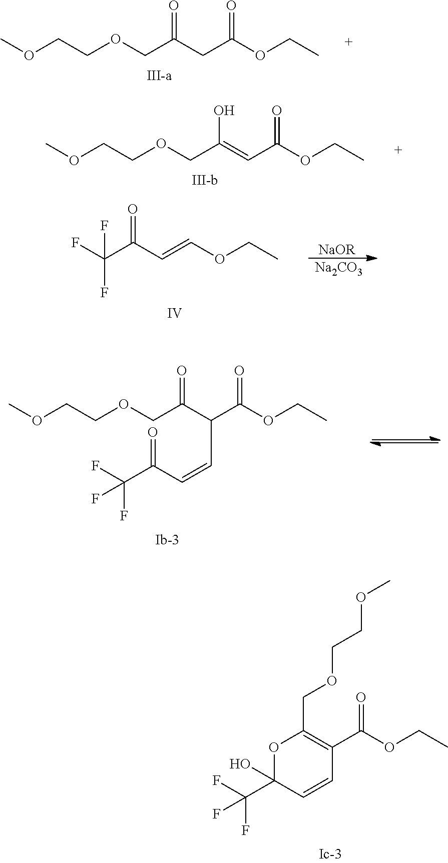

According to a specific embodiment of the present application, in step (1), a molar ratio of ethyl 4-chloroacetoacetate to the salt of 2-methoxyethanol is 1:1.8-2.5. The inventors of the present application have found through a research that in the case of a low proportion of the salt of 2-methoxyethanol, the raw materials are incomplete in reaction; and a high proportion results in side reactions, which in turn results in a decrease in the yield. Therefore, the optimal molar ratio is 1:2-2.3. It is deduced backwards therefrom, in the preparation of the salt of 2-methoxyethanol, a molar ratio of the base to the compound as shown in the formula IV is 1-3:1, optionally 2-2.5:1, preferably 2-2.3:1.

Further, in step (1), the material containing a salt of 2-methoxyethanol is a reaction material obtained by a reaction of 2-methoxyethanol under the action of a base.

Further, a reaction temperature for 2-methoxyethanol and the base is 40-180° C., or 80-150° C., or 80-130° C.

Further, the base is slowly added, and is added dropwise in the case of being a solution.

Further, the base is one or more selected from the group consisting of an organic base, an inorganic base, sodium hydride or metal sodium; the organic base includes one or more of sodium alkoxide and potassium alkoxide, preferably, the organic base includes one or more of sodium methoxide, sodium ethoxide, potassium tert-butoxide, potassium methoxide and potassium ethoxide; and the inorganic base includes one or more of sodium hydroxide, potassium hydroxide and sodium amide; preferably the base is one or more selected from the group consisting of sodium ethoxide, sodium methoxide, potassium methoxide and potassium ethoxide; and selecting a proper base can make the reaction route have better selectivity, so that the yield of the product is increased.

Further, in step (2), a reaction temperature is −15° C. to 30° C., preferably, 0° C.-10° C.

Further, in step (3), a reaction temperature is 0° C.-80° C., optionally, 30° C.-80° C., and preferably, 45° C.-60° C.

Further, in step (3), the ammonium salt includes one or more of ammonium chloride, ammonium carbonate, ammonium bicarbonate, ammonium nitrate, ammonium sulfate, ammonium phosphate, and ammonium acetate, preferably, the ammonium salt is ammonium acetate; and the ammonia exists in the form of ammonia gas and/or ammonia water.

Further, in step (3), a molar ratio of a compound as shown in the formula Ib-3 to the ammonium salt and/or the ammonia is 1: 1-5, optionally, 1: 1-2.5, and preferably, 1:1.2-1.5.

Further, in step (2), the reaction material further includes at least one of an impurity compound C, an impurity compound D and an impurity compound E,

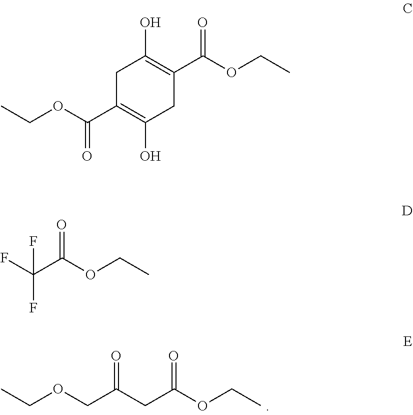

In the embodiment with the one-pot method of the reaction route C-1, the reaction material in step (1) is directly used for the reaction in step (2).

In the embodiment with the one-pot method of the reaction route C-1, the reaction material in step (2) is directly used for the reaction in step (3).

Generally, the intermediate I is conducive to formation of a kinetic product at a low temperature; while in the case of a high temperature, it is conducive to formation of a thermodynamic product.

The experiment shows that the intermediate I having the cis structure is conducive to the subsequent ring-closure reaction of the intermediate II; while a trans structure (enol) is difficult in ring closure, and requires a higher temperature. For example, if IC has a trans structure, due to the steric hindrance of the side chain and high activation energy of double bond inversion required by ring closure, ring closure cannot be achieved smoothly in a low temperature range.

A complete reaction route for preparing the intermediate II with the reaction routes A-1, B-1 and C-1 is as follows:

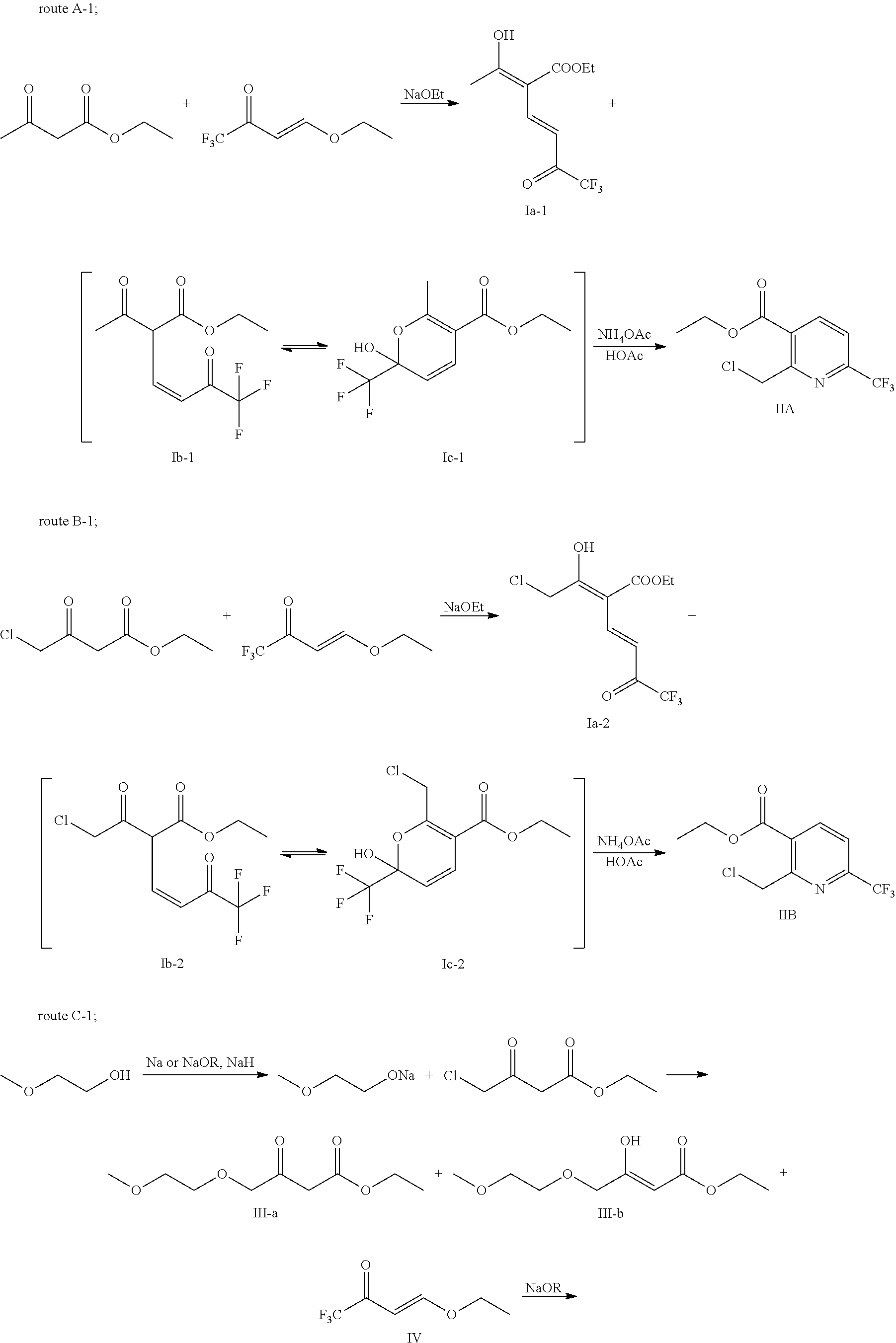

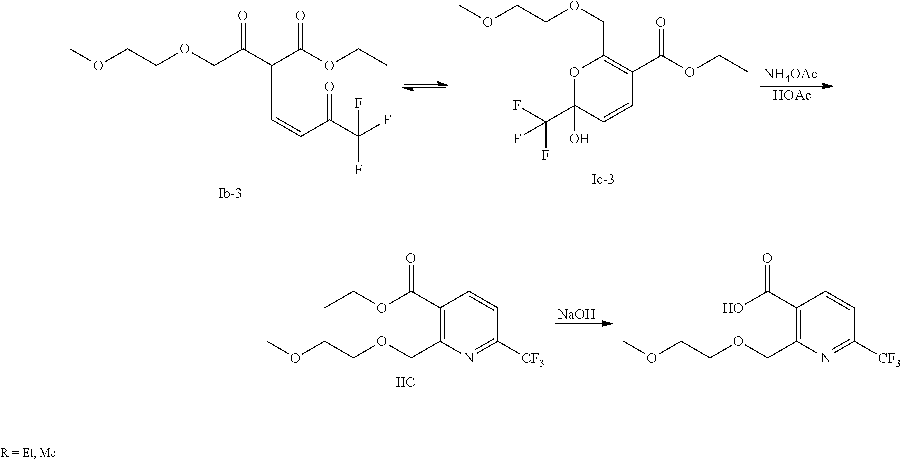

In the above route C-1 for the preparation of IIC, the intermediate Ib-3 can be prepared by a stepwise method, that is, the intermediates in each step can be purified by vacuum distillation before a next reaction (in the stepwise method, the keto cis Ib-3 is easily isomerized into hemiketal Ic-3 in the acid quenching reaction.). It is to be noted that if a temperature for vacuum distillation is too high, the keto cis Ib-3 or the hemiketal Ic-3 can be converted to enol trans Ia-3, resulting in subsequent failure of ring closure. A one-pot method may also be used, that is, reaction liquids obtained after each intermediate reaction are directly or simply treated and applied to a next reaction without purification, the advantage of which is that it avoids the formation of Ia-3 due to an unnecessary high temperature or post-treatment during an acid quenching reaction.

There are 4 methods for the preparation of a sodium salt of 2-methoxyethanol in the above route C-1 for the preparation of IIC. The first method is that adding 60 wt % of sodium hydride to toluene for reacting with 2-methoxyethanol; wherein a large amount of or excessive sodium hydride is often used; due to inclusion of mineral oil, a large amount of unreacted sodium hydride is often left in the system; and the quenching reaction can release a large amount of hydrogen, so that the method is not suitable for industrial production. The second method is that adding sodium hydroxide to excessive 2-methoxyethanol, and performing co-boiling with toluene for removing water in the reaction. The method has the advantages of cheap raw materials, a safe reaction and capability of mass production, yet has the disadvantages that the water is difficult to remove cleanly, and the product, the sodium salt of 2-methoxyethanol, is dark. The inventors of the present application have found that even trace water can affect subsequent reactions. The third method is a metal sodium method, in which the sodium salt is prepared by a reaction of metal sodium with 2-methoxyethanol.

The method is cheap in raw materials, controllable in production, and suitable for continuous production. Due to the release of a large amount of hydrogen, there are safety hidden dangers. The fourth method is an sodium alkoxide exchange method, that is, alcohol with a low boiling point is evaporated by the reaction of sodium methoxide or sodium ethoxide with 2-methoxyethanol. The method can be enlarged, but has the disadvantage of a high cost due to a large usage amount of sodium alkoxide. Among the above four preparation processes of the sodium salt of 2-methoxyethanol, sodium alkoxide is preferred as the base, especially sodium methoxide and sodium ethoxide.

Compared with the stepwise method, the one-pot method is simple in treatment and can be performed continuously. On one hand, it can reduce a loss caused by a separation process. On the other hand, a corresponding yield of the product is high, but a content of the product is low. The stepwise method has the advantages that the by-products produced in each step of the reaction can be removed by distillation, and the yield is slightly low; however, the content of the product is high, which is conducive to subsequent crystallization of nicotinic acid.

However, it should be noted that the intermediate Ib-3 is poor in stability, such as storage stability. Ib-3 is easily isomerized upon acidification or at a high temperature (most Ib-3 is isomerized into Ic-3, and a small part is isomerized into Ia-3). Ic-3 is conducive to subsequent reactions, while Ia-3 cannot be used for subsequent reactions. Therefore, compared with the stepwise method, in the one-pot method, Ib-3 produced under the base condition does not require an acid quenching reaction, and the reaction material directly undergoes subsequent reactions, which can effectively avoid or reduce isomerization of the intermediate Ib-3 into Ia-3. Considering the reliability of the process, the one-pot method is superior to the stepwise method, so the process of the route C-1 takes the one-pot method as a preferred solution.

It should be noted that the present application has no special limitation on a post-treatment method in the above substitution reaction or ring-closure reaction. The post-treatment method can be performed with reference to conventional methods in the art, such as extraction, column chromatography, high-pressure preparation and crystallization.

### Beneficial Effects

(1) In the present application, two compounds for preparing nicotinic acid fragments are docked first under the action of a base to produce an intermediate I, and then the intermediate I is subjected to intramolecular ring closure by an ammonium salt, which can remarkably increase the yield of the bicyclopyrone intermediate (II), reduce side reactions, and overcome the defects in the prior art (for example, methods reported in WO2006059103 and WO2004078729A1) that a reaction of raw materials is easily incomplete due to intramolecular ring closure directly by an ammonium salt. In the present application, the stepwise method of producing an intermediate I by base hydrolysis and then performing a ring-closure reaction to produce a bicyclopyrone intermediate (II), is conducive to purification of the nicotinic acid in the subsequent step; while the one-pot method can reduce side reactions, and can further increase the yield.
(2) The present application can make selection among three reaction routes A-1, B-1, and C-1 according to raw materials, the intermediate I (Ia-1 and Ib-1, or Ia-2 and Ib-2, or Ib-3) is prepared first, and then the intermediate II is prepared, which is of great significance to reduce production of by-products, achieve large-scale production and reduce the production cost; and the intermediate II can be obtained with a high conversion rate and a high yield. Among them, the route C-1 is high in a linear yield, few in by-products and more suitable for industrial large-scale production. The route C-1 is a preferred route due to few side reactions, for example, avoiding the disadvantages in the route A-1 of poor selectivity of methyl halogenation and the disadvantages in the route B-1 of more side reactions of the ethyl 4-chloroacetoacetate. In the two solutions of the route C-1, the one-pot method can avoid the risk of converting the cis intermediate Ib-3 into the trans intermediate Ia-3 due to separation/purification in the stepwise method. Therefore, the one-pot method in the route C-1 is the preferred solution.

### BRIEF DESCRIPTION OF THE DRAWINGS

One or more embodiments are exemplarily described by drawings in corresponding accompanying drawings, and such exemplary description does not constitute a limitation on the embodiments. The specific term “exemplary” used here means “serving as an example, embodiment or illustration”.

Any embodiment described herein as “exemplary” is not necessarily to be construed as advantageous over other embodiments.

FIG. 1 is a nuclear magnetic H spectrum of an analytical sample in step 1 of Example 1 of the present application;

FIG. 2 is a nuclear magnetic C spectrum of an analytical sample in step 1 of Example 1 of the present application;

FIG. 3 is a nuclear magnetic H spectrum of analytical samples in step 2) of step 1 of Example 2-3 of the present application;

FIG. 4 is a nuclear magnetic C spectrum of analytical samples in step 2) of step 1 of Example 2-3 of the present application;

FIG. 5 is a nuclear magnetic H spectrum of an impurity compound A in step 3) of step 1 of Example 2-3 of the present application;

FIG. 6 is a nuclear magnetic C spectrum of an impurity compound A in step 3) of step 1 of Example 2-3 of the present application;

FIG. 7 is a nuclear magnetic H spectrum of an impurity compound B in step 3) of step 1 of Example 2-3 of the present application;

FIG. 8 is a nuclear magnetic C spectrum of an impurity compound B in step 3) of step 1 of Example 2-3 of the present application;

FIG. 9 is a nuclear magnetic H spectrum of an intermediate IIB in step 2 of Example 2-3 of the present application;

FIG. 10 is a nuclear magnetic C spectrum of an intermediate IIB in step 2 of Example 2-3 of the present application;

FIG. 11 is a nuclear magnetic H spectrum of intermediates III-a and III-b in step 1 of Example 3 of the present application;

FIG. 12 is a nuclear magnetic C spectrum of intermediates III-a and III-b in step 1 of Example 3 of the present application;

FIG. 13 is a nuclear magnetic H spectrum of an intermediate IC in step 2 of Example 3 of the present application;

FIG. 14 is a nuclear magnetic C spectrum of an intermediate IC in step 2 of Example 3 of the present application;

FIG. 15 is a nuclear magnetic H spectrum of an intermediate IIIC in step 4 of Example 4-3 of the present application;

FIG. 16 is a nuclear magnetic C spectrum of an intermediate IIIC in step 4 of Example 4-3 of the present application;

FIG. 17 is a nuclear magnetic H spectrum of an impurity compound E of Example 13 of the present application;

FIG. 18 is a nuclear magnetic C spectrum of an impurity compound E of Example 13 of the present application;

FIG. 19 is a nuclear magnetic H spectrum of an impurity compound C of Example 14 of the present application; and

FIG. 20 is a nuclear magnetic C spectrum of an impurity compound C of Example 14 of the present application.

### DETAILED DESCRIPTION OF THE INVENTION

In order to make objectives, technical solutions, and advantages of examples of the present application clearer, the technical solutions in the examples of the present application are described clearly and completely in the following. Apparently, the described examples are only part rather than all of the examples of the present application. On the basis of the examples of the present application, all other examples obtained by a person of ordinary skill in the art without making creative efforts shall fall within the scope of protection of the present application.

In addition, in order to better illustrate the present application, the following specific implementations are given in many specific details. It is understood to those skilled in the art that the present application can also be implemented without some specific details. In some examples, the raw materials, solutions, methods and means familiar to those skilled in the art have not been described in detail as not to unnecessarily obscure aspects of the examples of the present application.

In the entire description and claims, unless otherwise expressly stated, the term “comprise” or the transformation of the term, such as “contain” or “include”, will be understood to include the stated elements or components, and not to exclude other elements or components.

Contents of products in the following examples are confirmed by a liquid or gas chromatograph to facilitate calculation of a yield.

In the following examples, in order to make intermediate I correspond to and distinguish from a reaction route, the intermediate I is named intermediate IA (corresponding tautomers Ia-1, Ib-1 and/or Ic-1), intermediate IB (corresponding tautomers Ia-2, Ib-2 and/or Ic-2) or intermediate IC (corresponding structural formulas Ib-3 and/or Ic-3).

In the following examples, GC-MS refers to gas chromatography-mass spectrometry, LC-MS refers to liquid chromatography-mass spectrometry, GC detection refers to gas chromatography detection, and HPLC detection refers to liquid chromatography detection.

### Example 1

### Synthesis of ethyl 2-methyl-6-(trifluoromethyl) nicotinate (intermediate IIA) (one example of route A-1)

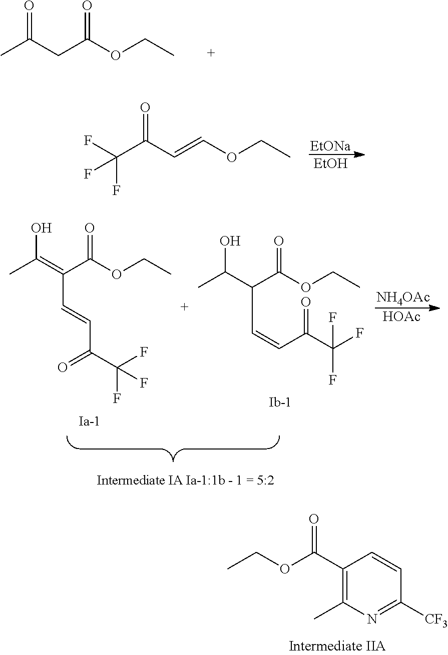

### Step 1: Synthesis of Intermediate IA (enol Ia-1; keto Ib-1)

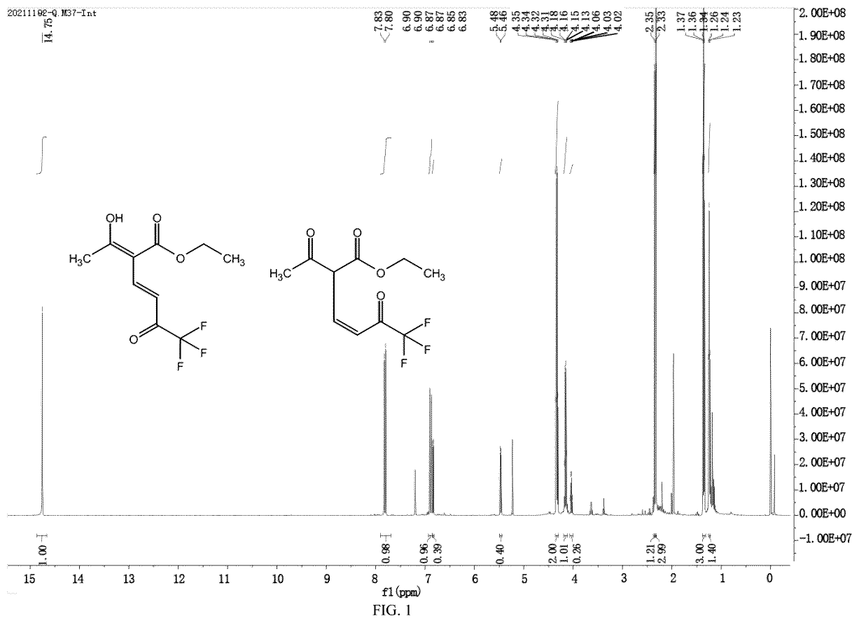

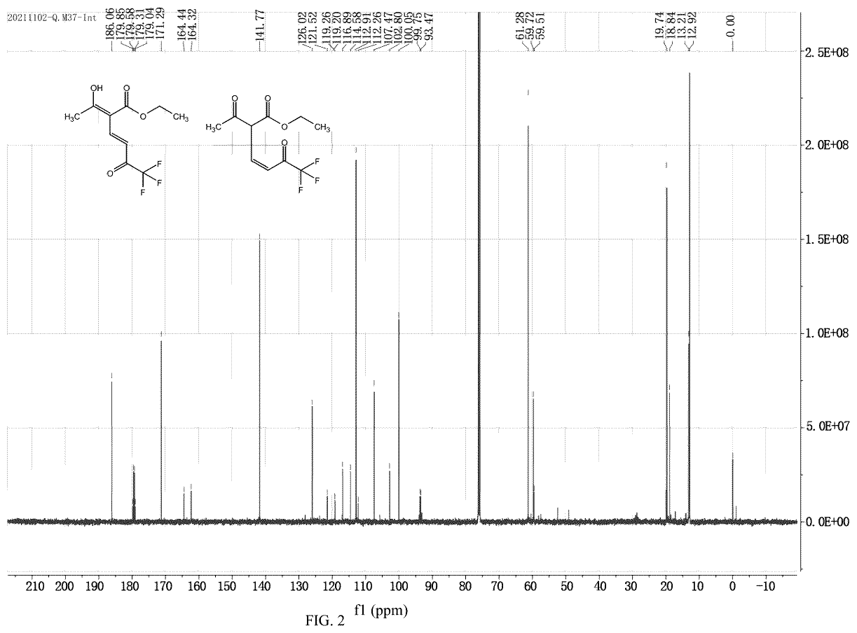

Ethyl acetoacetate (6 g, 46.5 mmol) and 4-ethoxy-1,1,1-trifluorobut-3-en-2-one (8 g, 47.6 mmol) were added to a single-necked flask, and cooled in an ice water bath. An ethanol solution of sodium ethoxide (56 mmol) was slowly added until complete dropping, stirring was performed at 0° C. for 2 h, monitoring was performed by thin layer chromatography (TLC) until an end of a reaction. The reaction liquid was poured into 50 mL of dilute hydrochloric acid; a resultant was extracted with ethyl acetate for three times (60 mL×3); organic phases were combined, and washed with saturated salt water; an organic phase was separated out, and concentrated to obtain 11.96 g of brown liquid; and the brown liquid was purified by column chromatography to obtain an analytical sample. The analytical sample is subjected to nuclear magnetic H and C spectral analysises, and results are shown in FIG. 1 and FIG. 2.

Nuclear magnetic H and C spectral analysises on the intermediate IA (enol Ia-1; keto Ib-1) (FIG. 1 and FIG. 2) are as follows:

LC-MS: M+1=253, M−1=251

¹H NMR (CDCl₃, 500 MHz), δ(ppm): (enol) 14.75 (s, 1H), 7.82 (d, 1H, J=15.0 Hz), 6.89 (d, 1H, J=15.0 Hz), 4.33 (q, 2H, J=5.0 Hz), 2.33 (s, 3H), 1.36 (t, 3H, J=5.0 Hz); (ketone) 6.85 (d, 0.4H, J=10.0 Hz), 5.47 (d, 0.4H, J=10.0 Hz), 4.16 (q, 1H, J=5.0 Hz), 4.02-4.06 (q, 0.2H, J=5.0 Hz), 2.35 (s, 1.2H), 1.24 (t, 1.2H, J=5.0 Hz)

¹³C NMR (CDCl₃, 150 MHz), δ (ppm): 186.06, 179.45 (q, J_{C-F}=40.5 Hz), 171.29, 164.44, 162.32, 141.77, 126.02, 121.52, 119.26, 115.79 (q, J_{C-F}=346.5 Hz), 112.91, 107.47, 102.80, 100.05, 93.62 (q, J_{C-F}=19.5 Hz), 61.28, 59.72, 19.74, 18.84, 13.21, 12.92

Mass spectrometry data shows that a molecular weight of the intermediate IA is 252, which is equivalent to a molecular weight of the structural formula IA (Ia-1, Ib-1). Nuclear magnetic hydrogen spectrum data shows that δ14.76 ppm in the formula Ia-1 contains an active hydrogen, having the features of a phenolic hydroxyl group, and forms a hydrogen bond with a neighboring atom in space, which is an enol hydroxyl hydrogen. Coupling constants of δ7.82 ppm and δ6.89 ppm are J=15.0 Hz, which proves that these two hydrogens are trans olefinic bonds; while coupling constants of δ6.84 ppm and δ5.47 ppm in the formula Ib-1 are J=10.0 Hz, which proves that the two hydrogen are cis olefinic bonds. In a carbon spectrum, δ186.06 ppm and δ179.45 ppm split into quartets, indicating that they are carbon attached to CF₃; δ115.79 ppm is a quartet, and J_{C-F}=346.5 Hz, indicating that δ115.79 ppm is carbon in CF₃.

### Step 2: Synthesis of ethyl 2-methyl-6-(trifluoromethyl) nicotinate (intermediate IIA)

The brown liquid (11.4 g) prepared in step 1 was dissolved into acetic acid (20 mL); a resultant was stirred at a room temperature; ammonium acetate (4.28 g) was added for stirring for about 0.5 h; then a temperature was raised to 50° C. for continuing to react for 1.5 h; and the system became brownish red. The acetic acid was recovered by vacuum concentration at 70° C.; a residue was extracted for three times with 150 mL of dichloromethane; organic phases were combined and washed with a small amount of saturated sodium bicarbonate aqueous solution; and the organic phase was separated out, and concentrated to obtain 10.8 g of brown oily matter with a content being 74.1%. A total yield of step 1 and step 2 is 77.4%.

In Example 1, the intermediate IA (enol Ia-1; keto Ib-1) is obtained under the action of a base by a stepwise method, and then the intermediate IIA is synthesized, which can remarkably reduce side reactions, improve the selectivity of the reaction, achieve a ring-closure reaction under a gentle condition, and increase the yield from 67% in the prior art to 77.4%.

### Example 2

### Synthesis of ethyl 2-chloromethyl-6-(trifluoromethyl) nicotinate (intermediate IIB)

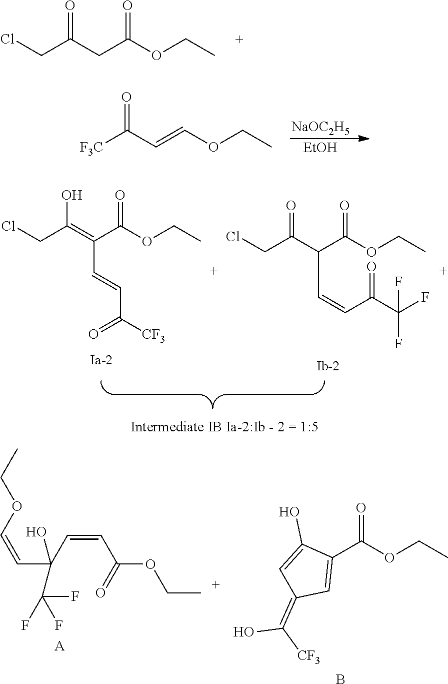

### Example 2-1

### Synthesis of Intermediate IB (enol Ia-2; keto Ib-2)

Anhydrous ethanol (20 g) and ethyl 4-chloroacetoacetate (3.46 g, 21 mmol) were added to a 250 mL four-necked flask, and 4-ethoxy-1,1,1-trifluorobut-3-en-2-one (3.36 g, 20 mmol) was added while stirring. A temperature was descended to −15° C., and sodium ethoxide (2.04 g, 30 mmol) dissolved into ethanol was slowly added dropwise with dropping completed for about 1.0 h. The temperature was kept at −15° C. for about 2.0 h, and monitoring was performed with thin layer chromatography (TLC) until an end of the reaction. A reaction liquid was poured into 30 mL of prepared dilute hydrochloric acid; the ethanol was removed by rotary evaporation; an aqueous phase was extracted with ethyl acetate (10 mL×3) for three times; organic phases were combined, dried with anhydrous magnesium sulfate, and subjected to rotary evaporation and concentration to obtain 4.25 g of crude product with a yield being 54.3%.

### Example 2-2

### Synthesis of Intermediate IB (enol Ia-2; keto Ib-2)

Anhydrous ethanol (35 g), ethyl 4-chloroacetoacetate (17.0 g, 102 mmol) and 4-ethoxy-1,1,1-trifluorobut-3-en-2-one (16.8 g, 100 mmol) were added to a 250 mL four-necked flask. A temperature was raised to 30° C., and sodium ethoxide (7.0 g, 102.9 mmol) dissolved into ethanol was slowly added dropwise with dropping completed for about 1.0 h. The temperature was kept at 30° C. for about 2.0 h, and the reaction was middle controlled by LC to stop when the content of the raw materials was smaller than 1%. A reaction liquid was poured into 100 mL of dilute hydrochloric acid; a pH value was regulated to 1-2; an aqueous phase was extracted with ethyl acetate (100 mL×3) for three times; organic phases were combined, dried with anhydrous magnesium sulfate, and subjected to rotary evaporation and concentration to obtain 29.0 g of crude product with a yield being 67.7%.

### Example 2-3

### Synthesis of ethyl 2-chloromethyl-6-(trifluoromethyl) nicotinate (intermediate IIB)

### Step 1: Synthesis of Intermediate IB (enol Ia-2; keto Ib-2)

1) Anhydrous ethanol (28 g) and ethyl 4-chloroacetoacetate (17.63 g, 107 mmol) were added to a four-necked flask, and 4-ethoxy-1,1,1-trifluorobut-3-en-2-one (17.7 g, 105 mmol) was added while stirring. A temperature was descended to 0° C., and sodium ethoxide (7.16 g) dissolved into ethanol was slowly added dropwise with dropping completed for about 2.0 h. The temperature was kept at 0° C. for about 2.0 h, and the reaction was middle controlled by LC to stop when the content of the raw materials was smaller than 1%; a reaction liquid was poured into 100 mL of prepared dilute hydrochloric acid solution, making a pH value be 2-3; and a resultant was extracted with dichloromethane (150 mL). The aqueous phase was extracted with dichloromethane (100 mL×2) for two times; and organic phases were combined and washed with saturated salt water once, to separate out the organic phase. The organic phase was concentrated to obtain 32.62 g of crude product which was an orange-yellow liquid.

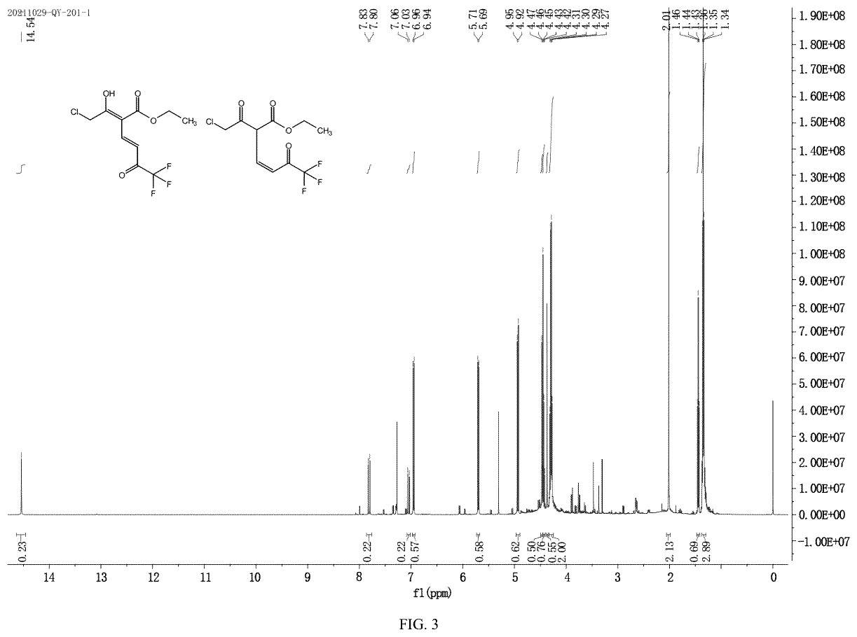

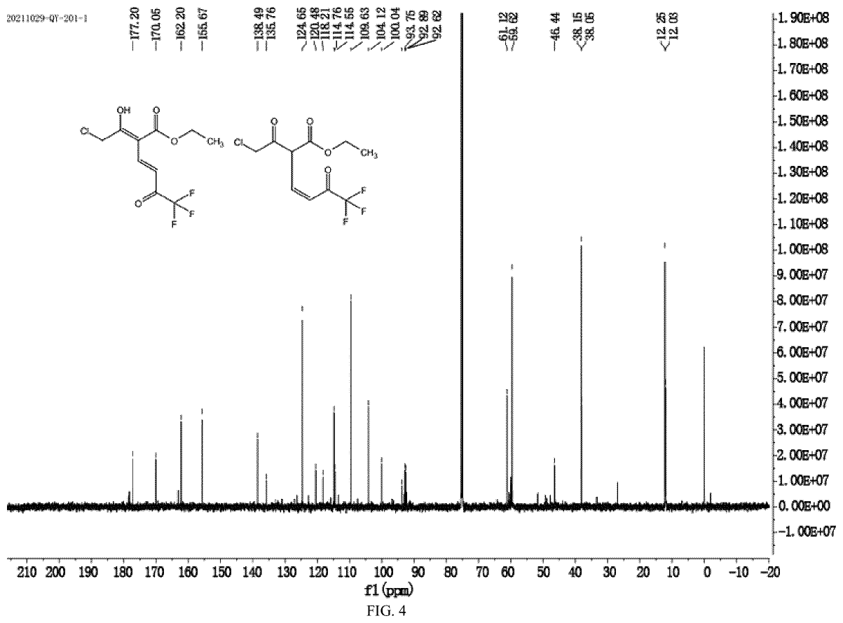

2) The crude product obtained by concentrating the organic phase in 1) was purified by column chromatography to obtain 21.8 g of light yellow pure product with a yield being 72.4%, where a ratio of the formula Ia-2 and the formula Ib-2 was about 1:5. The sample purified by column chromatography was subjected to nuclear magnetic H and C spectrum analysis, as shown in FIGS. 3 and 4.

3) For the concentrated crude product in 1), an impurity compound A (100 mg, a purity being 97%) and an impurity compound B (1.0 g, a purity being 95%) were obtained by preparing a liquid phase at a high pressure with acetonitrile and water as mobile phases.

Nuclear magnetic H and C spectral analysises on the intermediate I (enolIa-2, keto Ib-2) (FIG. 3 and FIG. 4) are as follows:

LC-MS: M+1=287

¹H NMR (CDCl₃, 500 MHz), δ(ppm): (compound Ia-2) 14.54 (s, 1H), 7.82 (d, 1H, J=15.0 Hz); 7.05 (d, 1H, J=15.0 Hz), 4.44 (q, 2H, J=5.0 Hz), 4.37 (s, 2H), 1.44 (t, 3H, J=5.0 Hz); (compound Ib-2) 6.95 (d, 1.25H, J=10.0 Hz), 5.70 (d, 1.25H, J=10.0 Hz), 4.93 (d, 1.25H, J=10.0 Hz), 4.46 (d, 1.25H, J=10.0 Hz), 4.27-4.31 (m, 3.75H), 2.01 (s, 2H), 1.35 (t, 3.75H, J=5.0 Hz)

¹³C NMR (CDCl₃, 150 MHz), δ (ppm): 177.20 (d, J_{C-F}=40.5 Hz), 170.05, 160.20, 155.67, 138.49, 135.76, 124.65, 119.35 (q, J_{C-F}=339.0 Hz), 114.76, 114.55, 109.63, 104.12, 100.04, 92.89 (d, J_{C-F}=42.0 Hz), 61.12, 59.62, 46.44, 38.15, 38.05, 12.25, 12.03

In this example, during the synthesis of the intermediate IB (enol Ia-2, keto Ib-2) in step 1, due to the action of the base, a plurality of sensitive groups present in the ethyl 4-chloroacetoacetate, so that the reaction is very complex; and there are still four main by-product impurity compounds A, B, C and D besides the intermediate I as a main product (enol Ia-2, keto Ib-2).

Formations of the impurity compound A, the impurity compound B, the impurity compound C and the impurity compound D are competitive with the intermediate IB as the main product (enol Ia-2, keto Ib-2), which is related to the addition mode of the base. In general, if the base is added quickly, due to a high concentration, 4-ethoxy-1,1,1-trifluorobut-3-en-2-one is subjected to acid decomposition to produce ethyl acrylate anions, and then addition is performed to produce the impurity compound A; while if the base is added slowly, due to a low overall concentration of the base, production of A is almost not detected. Formations of the impurity compound B and the impurity compound C are independent of a feeding method of the base and a type of the base. A content of B is about 6-12%, and a content of C is about 3% generally. The by-products B and C are inevitably produced, and are also main impurities in this example. A possible formation mechanism of the impurity compound A is as follows:

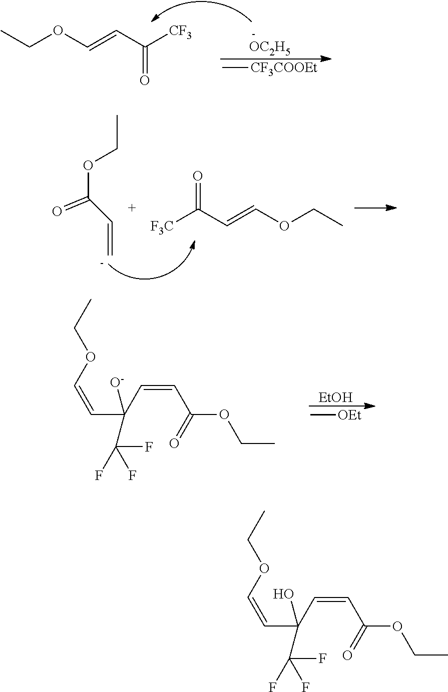

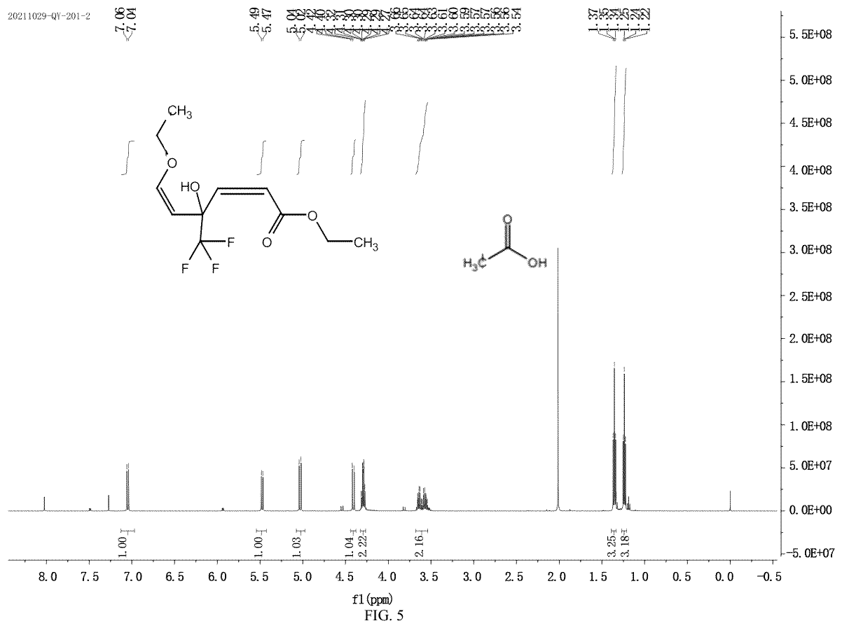

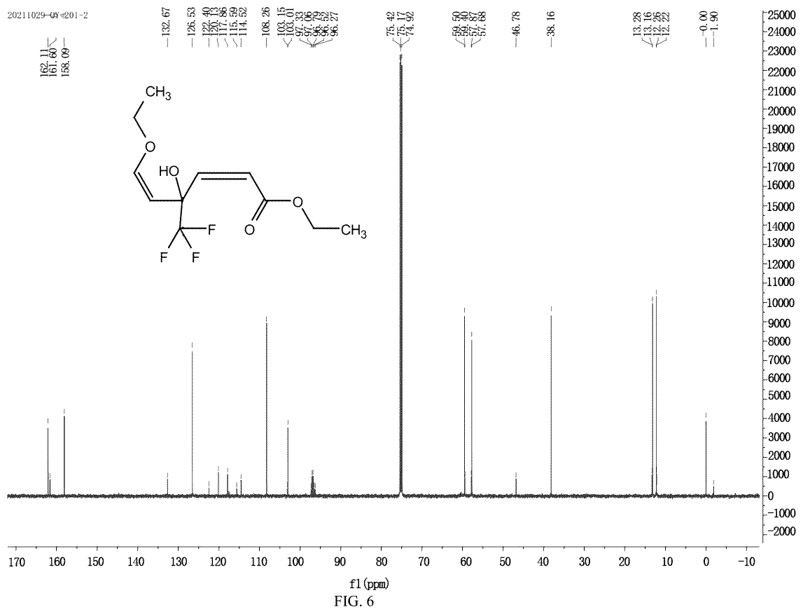

Nuclear magnetic H and C spectral analysises on the impurity compound A are shown in FIGS. 5 and 6.

LC-MS: M+1=269

¹H NMR (d6-DMSO, 500 MHz), δ (ppm): 7.05 (d, 1H, J=10.0 Hz), 5.48 (d, 1H, J=10.0 Hz), 5.03 (d, 1H, J=10.0 Hz), 4.41 (d, 1H, J=10.0 Hz), 4.27-4.32 (m, 2H), 3.56-3.66 (m, 2H), 1.35 (t, 3H, J=10.0 Hz), 1.24 (t, 3H, J=10.0 Hz)

¹³C NMR (d6-DMSO, 150 MHz), δ (ppm): 162.11, 158.09, 126.53, 119.00 (q, J_{C-F}=340.50 Hz), 108.26, 96.79 (t, J_{C-F}=40.5 Hz), 59.50, 57.68, 13.16, 12.26

Mass spectrometry data shows that a molecular weight of the impurity compound A is 268, which is equivalent to a molecular weight of the structural formula (impurity compound A). Hydrogen spectrum data shows that the molecular structure contains 2 ethoxy groups and 4 olefinic hydrogens, wherein the 4 olefinic hydrogens are on different olefinic bonds. A chemical shift of δ162.11 ppm in the carbon spectrum is carbonyl carbon, δ119.00 ppm splits into a quartet and J_{C-F}=340.50 Hz, indicating that the molecule contains CF₃; 696.79 ppm splits into a triplet, and J_{C-F}=40.5 Hz, indicating that the carbon is directly attached to CF₃, where a CH₂ at δ3.56-3.66 ppm splits into two groups, indicating that the structure has a chirality, and is a pair of racemic isomers.

A formation mechanism of the impurity compound B is as follows:

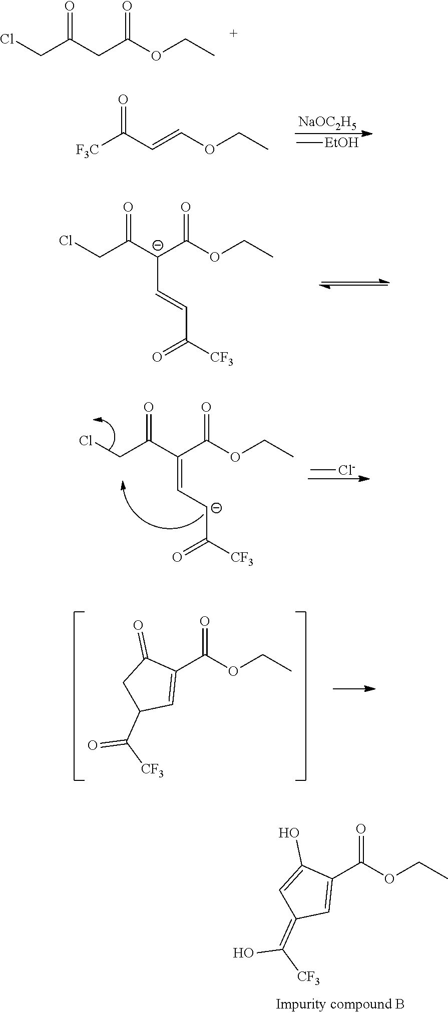

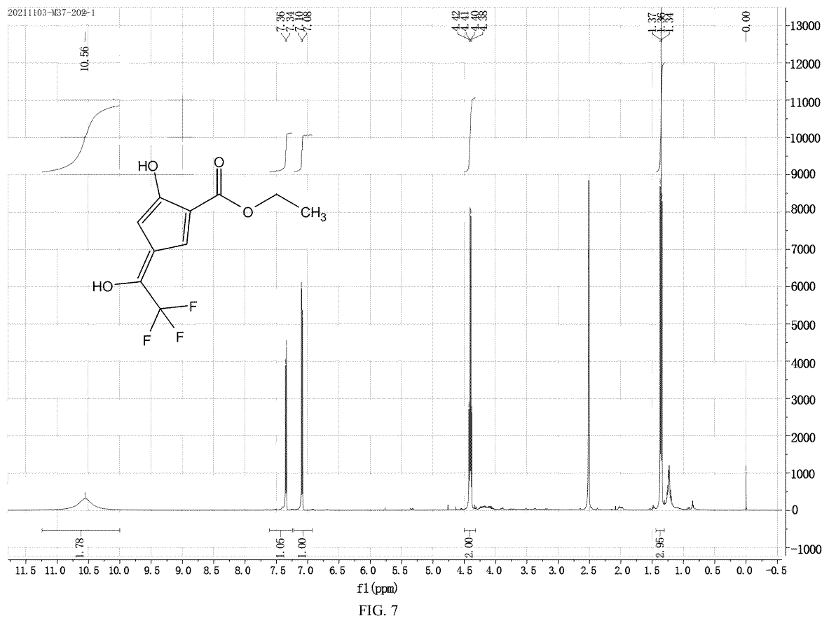

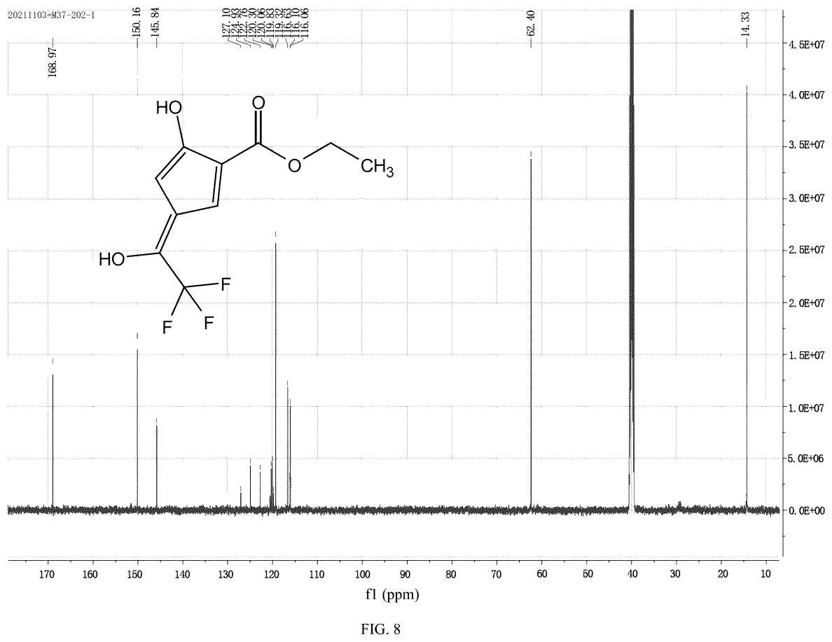

Nuclear magnetic H and C spectral analysises on the impurity compound B are shown in FIGS. 7 and 8.

LC-MS: M+1=251, M−1=249

¹H NMR (d6-DMSO, 500 MHz), δ (ppm): 10.56 (brs., 2H), 7.35 (d, 1H, J=10.0 Hz), 7.09 (d, 1H, J=10.0 Hz), 4.41 (q, 2H, J=5.0 Hz), 1.36 (t, 3H, J=5.0 Hz)

¹³C NMR (d6-DMSO, 150 MHz), δ (ppm): 168.97, 150.16, 145.84, 123.85 (q, J_{C-F}=325.5 Hz), 119.95 (q, J_{C-F}=34.5 Hz), 119.32, 116.63, 116.08 (d, J_{C-F}=6 Hz), 62.40, 14.33

Mass spectrometry data shows that a molecular weight is 250, which is equivalent to a molecular weight of the structural formula (impurity compound B). Hydrogen spectrum data shows that a wide singlet δ 10.56 ppm contains 2 active hydrogens, having the features of a phenolic hydroxyl group, and forms hydrogen bonds with the phenolic hydroxyl group and neighboring atoms in space. In another aspect, δ 168.97 ppm shows only one carbonyl group. The molecule contains an ethoxy group, which is a carbonyl group of ester, indicating that the other two “carbonyl groups” in the molecule exist in an enol form rather than a keto form.

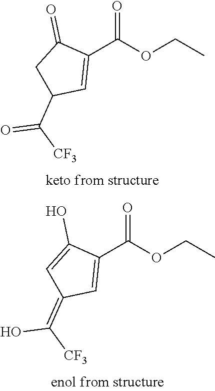 [SMILES: CCOC(=O)C1=CC(C(=O)C(F)(F)F)CC1=O ; CCOC(=O)C1=C/C(=C(/O)C(F)(F)F)C=C1O]

δ 7.35 ppm and δ 7.09 ppm are olefinic hydrogen, and color developing under UV indicates that the molecule should be aromatic; in the carbon spectrum, δ 123.85 ppm splits into a quartet, and J_{C-F}=325.5 Hz, indicating that it contains a CF₃ group; and δ 119.95 ppm splits into a quartet, and J_{C-F}=34.5 Hz, indicating that the carbon is attached to the CF₃ group, and is not carbonyl carbon.

There are also many other possible by-products in step 1 of this example (about 20 kinds) with a content generally smaller than 3%, for example:

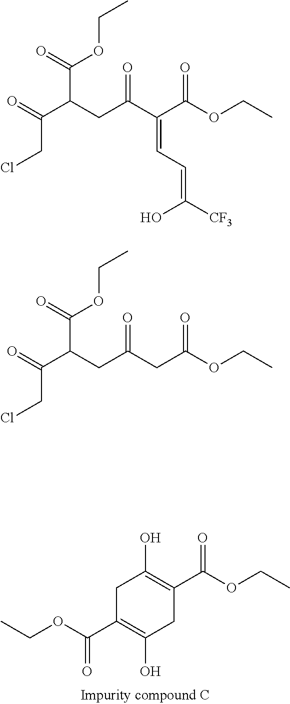

The main reason is that ethyl 4-chloroacetoacetate contains a plurality of active sites, and the selectivity of the reaction is not high. Even if the temperature is descended to −15° C., the reaction results are little affected. The content of the impurity compound C is about 3%. The reaction mechanism is as follows:

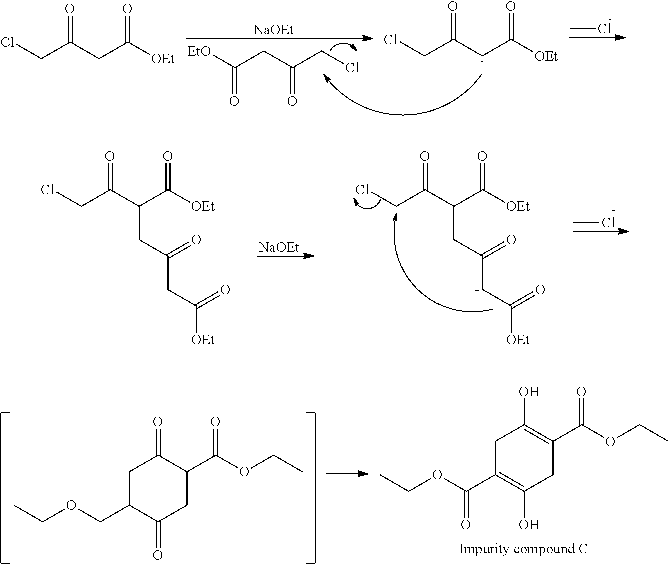

Mass spectrometry shows that a molecular weight of the impurity compound C is 256. It is speculated from the carbon spectrum and hydrogen spectrum data that a molecular structure is symmetrical. In the hydrogen spectrum, δ 12.13 ppm is enol hydrogen and forms intramolecular hydrogen bonds with neighboring groups. δ 170.29 ppm proves that there is only one kind of carbonyl carbon. The existence of the ethoxy group indicates that the structure contains only an ester group other than a ketone group.

In step 1 of Example 2, ethyl 4-chloroacetoacetate and 4-ethoxy-1,1,1-trifluorobut-3-en-2-one are used as substrates. In the process of adding sodium alkoxide dropwise, the sodium alkoxide plays a role of forming ethyl 4-chloro-3-oxobutanoate anions, which further reacts with 4-ethoxy-1,1,1-trifluorobut-3-en-2-one to form an intermediate IB (enol Ia-2 and keto Ib-2), and the intermediate IB undergoes an intramolecular reaction to form the impurity compound B; and the intermolecular reaction of ethyl 4-chloroacetoacetate produces the impurity compound C and other polymers. The effect of sodium alkoxide on 4-ethoxy-1,1,1-trifluorobut-3-en-2-one is to produce the impurity compound A and the impurity compound D by acid hydrolysis. This is the reason why the middle controlled raw materials completely disappear and the yield of the intermediate IB is low.

The intermediate IB formed in step 1 of this example has cis and trans tautomers with a ratio of about 1:5, where the trans isomer H has an enol structure. Due to formation of the intramolecular hydrogen bonds, the enol hydrogen moves toward a lower field to δ 14.54 ppm, and J=15.0 Hz, indicating that its structure is trans. The cis isomer I actually contains a chirality and a total of two olefinic hydrogen; and δ 1.34-1.36 at a higher field shows a multiplet instead of a simple triplet, indicating that it is a pair of diastereoisomers.

### Step 2: Synthesis of ethyl 2-chloromethyl-6-(trifluoromethyl) nicotinate (intermediate IIB)

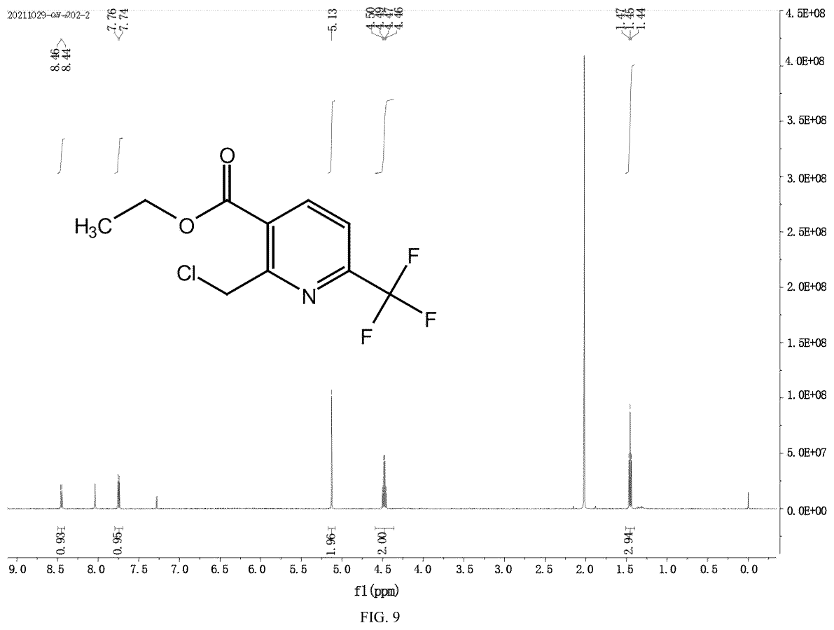

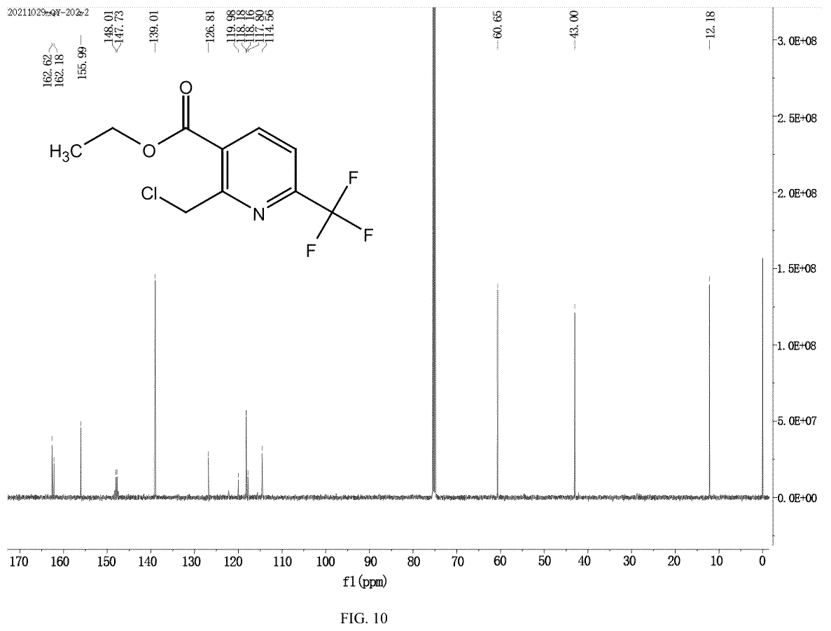

Acetic acid (130.48 g) and the product (18.17 g) in step 1 2) were added to a four-necked flask; ammonium acetate (9.44 g) was added while stirring; and a temperature was raised to 50° C. for heat preservation for 2 h. The reaction was middle controlled by LC to stop when the content of the raw materials was smaller than 1%. A solvent was removed by vacuum distillation; a residue was washed with a saturated sodium bicarbonate aqueous solution until no obvious bubble was produced; and dichloromethane (200 mL) was added for extraction. The aqueous phase was extracted with dichloromethane (100 mL×2); and organic phases were combined and washed with saturated salt water (100 mL) once. A solvent was removed by rotary evaporation to obtain 24.71 g of orange yellow crude product. The crude product was purified by column chromatography to obtain 14.3 g of faint yellow pure product (intermediate IIB) with the content being 95%, and the yield being 80.1%. Nuclear magnetic H and C spectral analysises on the pure product obtained by column chromatography are shown in FIGS. 9 and 10.

Nuclear magnetic H and C spectral analysises on the intermediate IIB are shown in FIGS. 9 and 10.

LC-MS: M+1=268

¹HNMR (CDCl₃, 500 MHz), δ (ppm): 8.45 (d, 1H, J=10.0 Hz), 7.75 (d, 1H, J=10.0 Hz); 5.13 (s, 2H), 4.48 (q, 2H, J=5.0 Hz), 1.45 (t, 3H, J=5.0 Hz)

¹³C NMR (CDCl3, 150 MHz), δ (ppm): 162.40 (d, J_{C-F}=66 Hz), 155.99, 147.87 (q, J_{C-F}=42 Hz), 139.01, 126.81, 118.89 (q, J_{C-F}=267 Hz), 116.37 (d, J_{C-F}=560 Hz), 60.65, 43.00, 12.18.

### Example 3

### Synthesis of ethyl 2-((2-methoxyethoxy)methyl)-6-(trifluoromethyl) nicotinate (intermediate IIC)

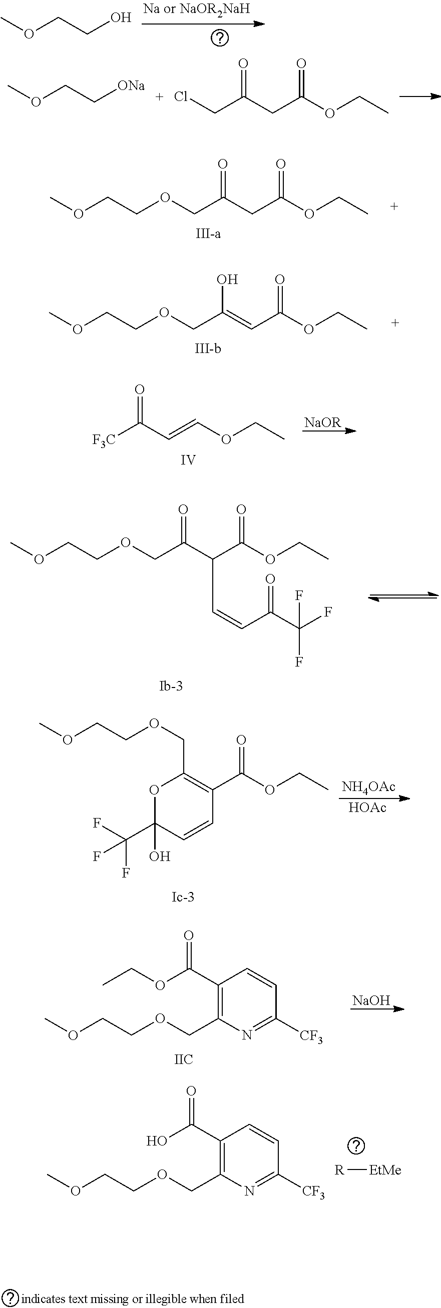

### Step 1 synthesis of ethyl 4-(2-methoxyethoxy)-3-oxobutyrate (i.e. IIIa) and ethyl (Z)-3-hydroxy-4 (2 methoxyethoxy)-3-oxobutyrate (i.e III-b)

Tetrahydrofuran (100 mL) was added in a four-necked flask and protected by nitrogen; and NaH (60%, 10.4 g, 260 mmol) was added in batches while stirring. The temperature was descended to 10° C., 2-methoxyethanol (21.6 g, 284 mmol) was slowly added dropwise, followed by production of bubbles; and after dropwise addition was completed, stirring was performed for 30 min. A mixed liquid of ethyl 4-chloroacetoacetate (10 g, 61 mmol) and tetrahydrofuran (50 mL) was added dropwise to the above four-necked flask; and after dropwise addition was completed, a reaction was performed at room temperature for 2 h. middle control was performed to stop the reaction when the residual ethyl 4-chloroacetoacetate was smaller than 1%. A solvent was removed under reduced pressure, and 150 mL of water was added to a residue; and a pH value was regulated with 30% HCl to 2-3. Dichloromethane (200 mL) was added for extraction, and the obtained aqueous phase was extracted with dichloromethane (100 mL×2) for two times; organic phases were combined, washed with saturated salt water once, dried with anhydrous magnesium sulfate, and concentrated to obtain 9.15 g of orange yellow liquid with a content being 73%, and a yield being 53.6%. A ratio of a keto form (i.e. corresponding to the structural formula III-a) to an enol form (i.e. corresponding to the structural formula III-b) is about 9:1.

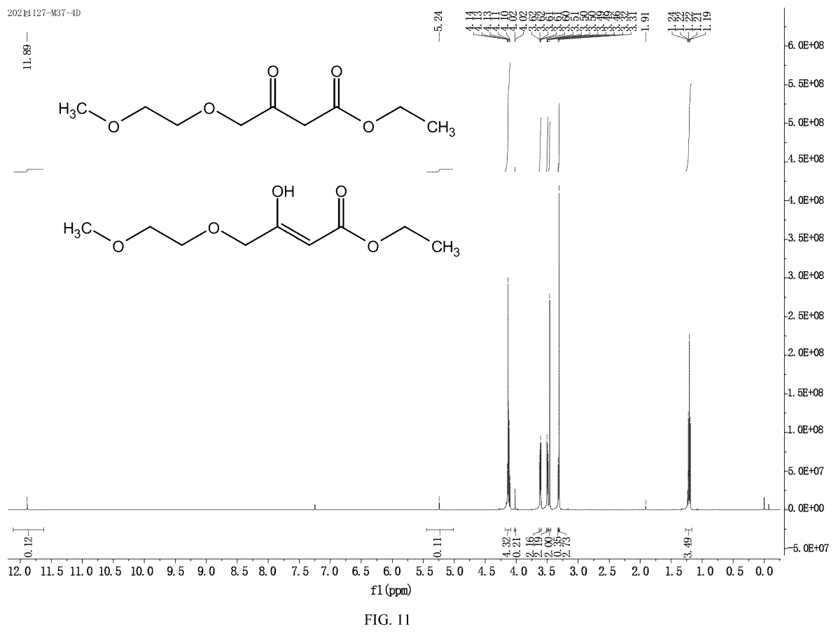

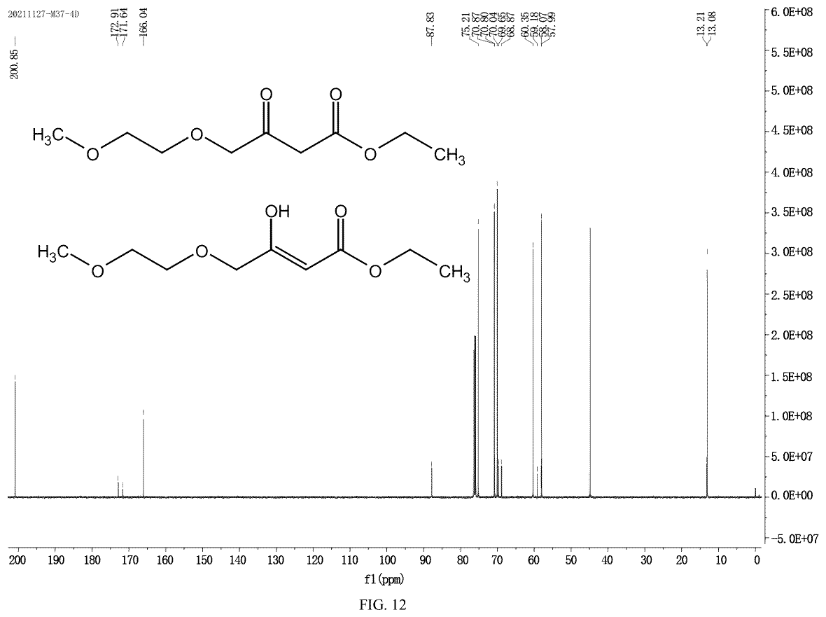

Nuclear magnetic H and C spectral analysises on the compounds III-a and III-b are shown in FIGS. 11 and 12.

GC-MS: M=204

¹H NMR (CDCl₃, 500 MHz), δ (ppm) (III-a): 4.10-4.14 (m, 4H), 3.61 (t, 2H, J=5.0 Hz), 3.50 (t, 2H, J=5.0 Hz), 3.46 (s, 2H), 3.31 (s, 3H), 1.21 (t, 3H, J=5.0 Hz); (III-b): 11.89 (s, 1H), 5.24 (s, 1H), 4.10-4.14 (m, 2H), 4.02 (s, 2H), 3.61 (t, 2H, J=5.0 Hz), 3.50 (t, 2H, J=5.0 Hz), 3.32 (s, 3H), 1.21 (t, 3H, J=5.0 Hz)

¹³C NMR (CDCl₃, 150 MHz), (III-a) δ: 200.85, 166.04, 75.21, 70.87, 70.04, 60.35, 57.99, 44.85, 13.08; (III-b) δ:172.91, 171.64, 87.83, 70.80, 69.65, 68.87, 59.18, 58.07, 13.21.

### Step 2: Synthesis of Intermediate IC

Ethanol (5.6 g) and 3.06 g of crude product (73%, 10.9 mmol) in step 1 were added to a four-necked flask, and (E)-4-ethoxy-1,1,1-trifluorobut-3-en-2-one (17.7 g, 105 mmol) was added while stirring.

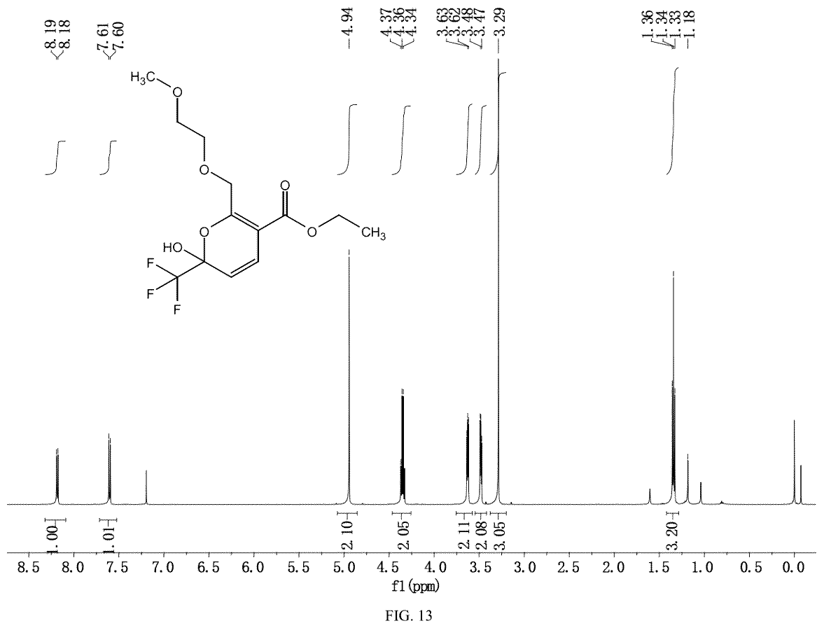

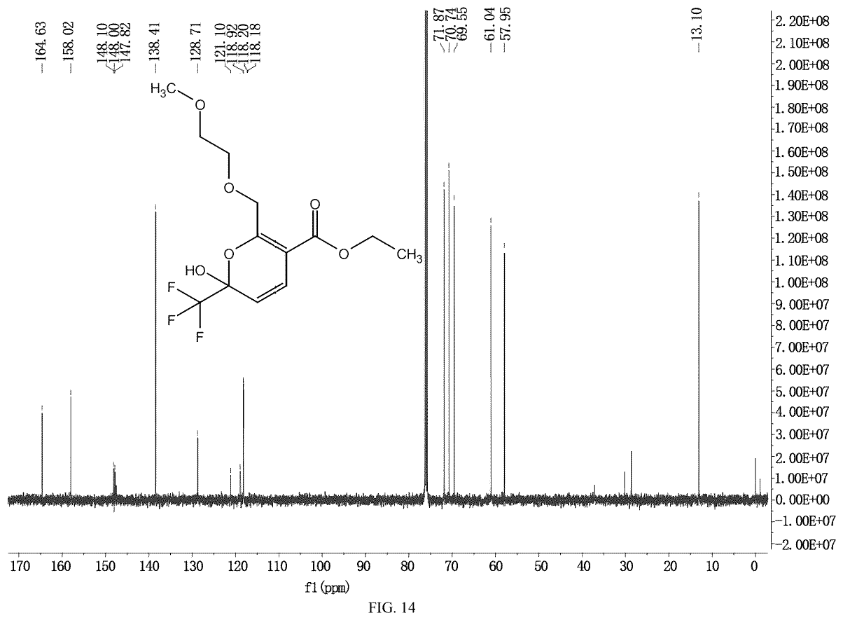

A temperature was descended to 0° C., and sodium ethoxide (0.72 g, 10.6 mmol) dissolved into ethanol was slowly added dropwise. After dropwise addition was completed, the temperature was kept for about 2.0 h, and the reaction was middle controlled by LC until the content of the raw materials was smaller than 1%. A reaction liquid was poured into 20 mL of prepared hydrochloric acid solution with a pH value being about 2-3. A resultant was extracted with dichloromethane (20 mL). An aqueous phase was extracted with dichloromethane (10 mL×2) for two times; and organic phases were combined. The combined organic phase was washed with saturated salt water once, to separate out the organic phase; the organic phase was dried with anhydrous magnesium sulfate, and concentrated to obtain 3.51 g of crude product which was an orange yellow liquid and was directly put into a next reaction without purification. Analytical samples were obtained by liquid phase preparation and separation, and subjected to nuclear magnetic H and C spectral analysises. Nuclear magnetic H and C spectral analysises on the intermediate IC are shown in FIGS. 13 and 14.

GC-MS: M=326

¹H NMR (CDCl₃, 500 MHz), δ (ppm): 8.19 (d, 1H, J=5.0 Hz), 7.61 (d, 1H, J=5.0 Hz), 4.94 (s, 2H), 3.35 (q, 2H, J=5.0 Hz), 3.63 (t, 2H, J=5.0 Hz), 3.48 (t, 2H, J=5.0 Hz), 3.29 (s, 3H), 1.34 (t, 3H, J=5.0 Hz)

¹³C NMR (CDCl₃, 150 MHz), δ (ppm): 164.43, 158.02, 148.05 (q, J_{C-F}=15.0 Hz), 138.41, 128.71, 120.01 (q, J_{C-F}=327.0 Hz), 118.20, 71.87, 70.74, 69.55, 61.04, 57.95, 13.10

### Step 3: Synthesis of ethyl 2-((2-methoxyethoxy)methyl)-6-(trifluoromethyl) nicotinate (intermediate IIC)

Acetic acid (14.04 g) and the crude product (3.51 g) in step 2 were added to a four-necked flask; ammonium acetate (0.94 g, 12.2 mmol) was added while stirring; and a temperature was raised to 50° C. for heat preservation for 2 h. The reaction was middle controlled by LC to stop when the content of the raw materials was smaller than 1%. A solvent was removed under reduced pressure; a residue was washed with a saturated sodium bicarbonate aqueous solution until no obvious bubble was produced; and dichloromethane (20 mL) was added for extraction. An aqueous phase was extracted with dichloromethane (15 mL×2); and organic phases were combined and washed with saturated salt water (15 mL) once. The organic phase was dried with anhydrous magnesium sulfate, and a solvent was removed under reduced pressure to obtain 3.75 g of crude product which was an orange yellow liquid with a content being 53%. A total yield of step 2 and step 3 is 59.3%. The total yield of step 1, step 2 and step 3 is 31.8%.

### Example 4

### Example 4-1

### Synthesis of ethyl 4-(2-methoxyethoxy)-3-oxobutyrate (i.e. III-a) and ethyl (Z)-3-hydroxy-4-(2-methoxyethoxy)-3-oxobutyrate (i.e. III-b)

Sodium ethoxide (6.8 g, 100 mmol) was added to 2-methoxyethanol (8.0 g, 105 mmol) for stirring and heating; a resultant was heated in an oil bath to 40° C. and stirred for 2 h; ethanol was removed by vacuum distillation; at this time, the system presents a brown yellow solid; the brown yellow solid was cooled to room temperature; and toluene (40.2 g) was added for stirring and dispersing to obtain a toluene solution of a sodium salt of 2-methoxyethanol.

The temperature was controlled at about 25° C., and ethyl 4-chloroacetoacetate (7.5 g, 45.5 mmol) was added to the toluene solution of the sodium salt of 2-methoxyethanol dropwise; after the addition was completed, the temperature was raised to 40° C.; stirring was performed for a reaction for 6 h; and TLC tracking was performed until the raw materials reacted completely. The temperature was descended to below 30° C.; a pH value of the reaction liquid was regulated with a hydrochloric acid solution to 4; stirring was performed for 10 min; a resultant was left for standing and liquid separation to separate out an organic phase; an aqueous phase was extracted with 30 mL of toluene; organic phases were combined; the toluene was removed by vacuum rotary evaporation to obtain a crude product of intermediate compounds III-a and III-b; and the crude product underwent vacuum evaporation with an oil pump to obtain 6.43 g of golden yellow product with a yield being 69.2%.

### Example 4-2

### Synthesis of ethyl 4-(2-methoxyethoxy)-3-oxobutyrate (i.e. III-a) and ethyl (Z)-3-hydroxy-4-(2-methoxyethoxy)-3-oxobutyrate (i.e. III-b)

Sodium ethoxide (6.8 g, 100 mmol) was added to 2-methoxyethanol (11.4 g, 150 mmol) for stirring and heating; a resultant was heated in an oil bath to 100° C. and stirred for 1 h, and continued to be heated to 180° C. to remove excessive 2-methoxyethanol by evaporation; at this time, the system presents a brownish black solid; the brownish black solid was cooled to room temperature; and toluene (40.2 g) was added for stirring and dispersing to obtain a toluene solution of a sodium salt of 2-methoxyethanol.

The temperature was controlled at about 25° C., and ethyl 4-chloroacetoacetate (7.5 g, 45.5 mmol) was added to the toluene solution of the sodium salt of 2-methoxyethanol dropwise; after the addition was completed, the temperature was raised to 40° C.; stirring was performed for a reaction for 6 h; and TLC tracking was performed until the raw materials reacted completely. The temperature was descended to room temperature; a pH value of the reaction liquid was regulated with a hydrochloric acid solution to 4; a resultant was stirred, and left for standing and liquid separation to separate out an organic phase; an aqueous phase was extracted with 30 mL of toluene; organic phases were combined; the toluene was removed by vacuum rotary evaporation to obtain a crude product of intermediate compounds III-a and III-b; and the crude product underwent vacuum evaporation with an oil pump to obtain 7.23 g of golden yellow product with a yield being 77.8%.

### Example 4-3

### Synthesis of ethyl 2-((2-methoxyethoxy)methyl)-6-(trifluoromethyl) nicotinate (intermediate IIC)

### Step 1: Synthesis of ethyl 4-(2-methoxyethoxy)-3-oxobutyrate (i.e. III-a) and ethyl (Z)-3-hydroxy-4-(2-methoxyethoxy)-3-oxobutyrate (i.e. III-b)

Sodium ethoxide (21.6 g, 317 mmol) was added to 2-methoxyethanol (72.6 g, 954 mmol) for stirring and heating; a resultant was heated in an oil bath to 130° C., and subjected to vacuum distillation to collect 67 g of a fraction; the temperature was descended to below 100° C.; and toluene (86.6 g) was added for stirring and dispersing to obtain a toluene solution of a sodium salt of 2-methoxyethanol.

The temperature was controlled at about 30° C., and ethyl 4-chloroacetoacetate (18.7 g, 114 mmol) was added to the toluene solution of the sodium salt of 2-methoxyethanol dropwise; after the addition was completed, the temperature was kept at 40° C.; stirring was performed for a reaction for 6 h; and TLC (PE:EA=6:1) tracking detection was performed until the raw materials reacted completely. The temperature was descended to below 30° C.; the reaction liquid was poured into a hydrochloric acid solution (112.2 g, 6.8%); a resultant was stirred for 10 min, and left for standing and liquid separation; an aqueous phase was extracted with toluene (43.2 g) for two times, and left for standing and liquid separation; the aqueous phase was extracted with dichloromethane (21.6 g) again; organic phases were combined and subjected to vacuum distillation (90° C., −0.095 MPa) to obtain a crude product of intermediate compounds III-a and III-b, which was a brown oily matter of 22.0 g. The crude product was heated in an oil bath to 130° C.; a solvent was evaporated under reduced pressure (−0.095 MPa) by a water pump until no distillate flowed out; an oil pump was replaced; an oil bath was heated to 140° C.; and 20.42 g of a golden yellow product was evaporated out with a yield being 87.6%, which was directly used in a next reaction.

By preparing a sodium salt of 2-methoxyethanol first and then preparing the compounds III-a and III-b in step 1 of Example 4, the yield may reach 87.6%, which is better than that obtained by the direct reaction in step 1 of Example 3 (53.6%).

### Step 2: Synthesis of Intermediate IC

The product (20.4 g, 100 mmol) obtained in the above step 1 and 4-ethoxy-1,1,1,1-trifluorobut-3-en-2-one (16.8 g, 100 mmol) were added to anhydrous ethanol (49.0 g); a temperature was controlled below 10° C.; and a 20 wt % ethanol solution of sodium ethoxide (34 g, 100 mmol) was added dropwise. After dropping was completed, the temperature was kept at 0° C.-10° C. for 2 h; and HPLC detection was performed until the raw materials reacted completely. The reaction liquid was poured into a mixed solution of 114.3 g of hydrochloric acid solution (prepared from 12.2 g of 30 wt % hydrochloric acid) and dichloromethane (81.7 g); a resultant was stirred for 10 min and left for standing and liquid separation; an aqueous phase was then extracted with dichloromethane (40.8 g); and organic phases were combined and subjected to vacuum distillation (55° C., below −0.095 MPa) to obtain an intermediate IC, which was a brown oily matter of 36.5 g and directly used for a next reaction.

### Step 3: Synthesis of ethyl 2-((2-methoxyethoxy)methyl)-6-(trifluoromethyl) nicotinate (intermediate IIC)

The intermediate IC (36.5 g) in step 2 was added to acetic acid (130.5 g), ammonium acetate (9.4 g, 122 mmol) was added while stirring; a temperature was controlled at 50° C.-60° C. for stirring reaction for 2 h; and HPLC detection was performed until the raw materials reacted completely. A mixed solution of water (97.9 g) and dichloromethane (97.9 g) was added to the reaction liquid; a resultant was stirred for 10 min and left for standing and liquid separation; an aqueous phase was then extracted with dichloromethane (65.2 g) for two times; and organic phases were combined and subjected to vacuum distillation (60° C., below −0.095 MPa) to obtain an intermediate IIC, which was a brown oily matter of 35.1 g and directly used for a next reaction.

### Step 4: Synthesis of 2-((2-methoxyethoxy)methyl)-6-(trifluoromethyl) nicotinate (intermediate IIIC)

The intermediate IIC (35.1 g, net content being 30.7 g) in step 3 was added to ethanol (30.7 g); a sodium hydroxide (42.7 g, 28%, 300 mmol) solution was added dropwise; a temperature was controlled at 50° C.-60° C. for stirring reaction for 1 h; and GC detection was performed until the raw materials reacted completely. The temperature was descended to room temperature; water (92.2 g) and dichloromethane (61.5 g) was added to the reaction liquid; a resultant was stirred for 10 min and left for standing and liquid separation; dichloromethane (92.2 g) was added to the aqueous phase; a resultant was acidized with 30% hydrochloric acid until a pH value was approximately equal to 1.5; stirring was performed for 10 min for liquid separation; then the aqueous phase was extracted with dichloromethane (61.5 g); and organic phases were combined and subjected to vacuum distillation (60° C., below −0.095 MPa) to obtain 26.5 g of an intermediate IIIC, which was a brown oily matter. 13.3 g of ethyl acetate and 16.6 g of petroleum ether were added to the obtained crude product; stirring was performed slowly and the temperature was descended to −5° C. for crystallization; and suction filtration and drying were performed to obtain a light yellow solid product of 15.2 g. The yield of steps 2, 3 and 4 is 54.5%.

The total yield of this example is 47.8%. The yield of step 2 and step 3 of Example 4 is high, which is significantly better than the total yield of Example 3 (the reaction yield of IIC in Example 3 is not too high, and no further preparation of IIIC is performed). The intermediate IIIC prepared in step 4 of Example 4 can be used for synthesizing bicyclopyrone.

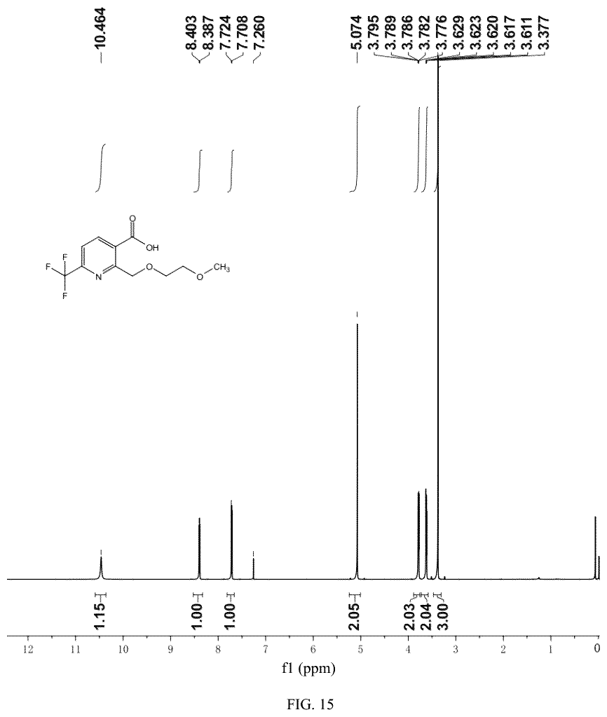

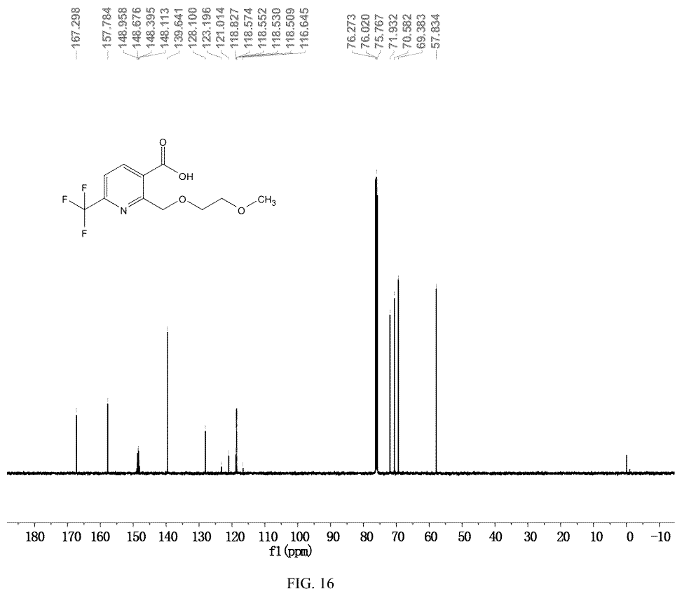

Nuclear magnetic H and C spectral analysises on the intermediate IIIC are shown in FIGS. 15 and 16.

LC-MS: M−1=278

¹H NMR (CDCl₃, 500 MHz), δ (ppm): 10.464 (s, 1H), 8.395 (d, 1H, J=8.0 Hz), 7.716 (d, 1H, J=8.0 Hz), 5.074 (s, 2H), 3.776-3.795 (m, 2H), 3.611-3.629 (m, 2H), 3.377 (s, 3H);

¹³C NMR (CDCl₃, 150 MHz), δ (ppm): 167.298, 157.784, 148.538 (q, J_{C-F}=42.3 Hz), 139.641, 128.100, 119.921 (q, J_{C-F}=327.3 Hz), 118.574, 71.932, 70.582, 69.383, 57.384.

### Example 5

### Synthesis of ethyl 2-((2-methoxyethoxy)methyl)-6-(trifluoromethyl) nicotinate (intermediate IIC) (one-pot method)

2-Methoxyethanol (19.1 g, 251 mmol) and toluene (82.7 g) were added to a four-necked flask; and metal sodium (5.3 g, 230 mmol) was added while stirring; a temperature was gradually raised; and a reaction was performed at 80° C. until no metal sodium particle was formed. The temperature was descended and controlled at below 40° C.; ethyl 4-chloroacetoacetate (16.5 g, 101 mmol) was added dropwise; the temperature was kept at 45° C. for reaction for about 3 h, and then descended. A sample was taken for GC detection to obtain a conversion rate of 94.6%; and the reaction liquid was directly used for a next reaction.

The above reaction liquid was cooled to below 10° C.; a mixed liquid of 4-ethoxy-1,1,1-trifluorobut-3-en-2-one (16.8 g, 100 mmol) and toluene (14.0 g) was added to the system; and after 2 hours of reaction, GC detection was performed to get no residual raw materials. Dilute hydrochloric acid was added for acidification while stirring; a resultant was left for standing and liquid separation; and a toluene phase was detected to have a content of 87.1%. The reaction liquid was directly used for a next reaction.

Ammonium acetate (9.3 g, 121 mmol) was added to the above toluene solution while stirring; the temperature was raised to 50° C. for reaction for 2 h; and HPLC middle control was performed, and the reaction was completed at about 3 h. A resultant was washed with water (50.0 g) for liquid separation; and 25.7 g of a toluene phase was weighed, and detected to have the content of 87.2%. The total yield of the intermediate IIC in three steps of this example is 72.9%.

### Example 6

### Synthesis of ethyl 2-((2-methoxyethoxy)methyl)-6-(trifluoromethyl) nicotinate (intermediate IIC) (one-pot method)

2-Methoxyethanol (19.1 g, 251 mmol) and toluene (82.7 g) were added to a four-necked flask; and metal sodium (5.1 g, 222 mmol) was added while stirring; a temperature was gradually raised to 80° C.; and a reaction was performed until no metal sodium particle was formed. The temperature was descended to below 40° C.; ethyl 4-chloroacetoacetate (16.5 g, 101 mmol) was added dropwise; the temperature was kept at 40° C. for reaction for about 6 h; reaction was performed at 65° C. for 2 h; and then the temperature was descended. A sample was taken for GC detection to obtain a conversion rate of 88.8%; and the reaction liquid was directly used for a next reaction.

The temperature of the system was cooled to below 10° C.; a mixed liquid of 4-ethoxy-1,1,1-trifluorobut-3-en-2-one (16.8 g, 100 mmol) and toluene (14.0 g) was added to the reaction liquid dropwise; and after 2 hours of reaction, GC detection was performed to get no residual raw materials. Dilute hydrochloric acid was added for acidification while stirring; a resultant was left for standing and liquid separation; and a toluene phase was detected to have a content of 75.3%. The reaction liquid was directly used for a next reaction.

Ammonia gas was introduced into the above toluene solution for 15 min while stirring; ammonium acetate (15.0 g) was added; the temperature was raised to 50° C. for reaction for 2 h; and HPLC middle control was performed, and the reaction was completed at about 3 h. A resultant was washed with water (50.0 g) for liquid separation; and 20.6 g of a toluene phase was weighed, and detected to have a content of 86.2%. The yield of the intermediate IIC in three steps of this example is 57.2%, and the reaction yield of this example is slightly low. The inventors of the present application speculate that it may be the reason for the use of the ammonia gas in the preparation of IIC.

### Example 7

### Synthesis of ethyl 2-((2-methoxyethoxy)methyl)-6-(trifluoromethyl) nicotinate (intermediate IIC) (one-pot method)

2-Methoxyethanol (114.4 g, 1.5 mol), sodium hydroxide (48.3 g, 1.21 mol) and toluene (250.3 g) were added to a four-necked flask; the four-necked flask was immersed into an oil bath at a high temperature of 140° C.-150° C. for refluxing; water produced by the reaction was removed with a water separator until there was no obvious water droplet in the water separator; the four-necked flask was cooled in an ice water bath to about 30° C.; and ethyl 4-chloroacetoacetate (90.61 g, 0.55 mol) was added dropwise; a reaction was performed at 40° C. overnight; a sample was taken for GC detection to get a conversion rate of 95.9%; and a reaction liquid was directly used for a next reaction.

A part of the reaction liquid was taken, and folded to contain a 2-methoxyethanol sodium salt (0.1 mol); a temperature was controlled to below 10° C.; a toluene solution of 4-ethoxy-1,1,1-trifluorobut-3-en-2-one (containing 16.8 g of 4-ethoxy-1,1,1-trifluorobut-3-en-2-one and 16.89 g of toluene) was added dropwise; after 2 hours of reaction, GC detection was performed until there was no residual raw materials; and dilute hydrochloric acid was added for acidification while stirring; a resultant was left for standing and liquid separation; a toluene phase was detected to have a content of 82.7%; and the reaction liquid was directly used for a next reaction.

Ammonium acetate (9.25 g, 0.12 mol, equally divided into three parts, and adding one batch every 20 min) was added to the above toluene solution in batches while stirring; the temperature was raised to 50° C. for reaction for 2 h; a HPLC middle control was performed; after the reaction was completed, water (80 g) was added for washing and liquid separation; and the toluene phase was weighed as 17.4 g, and detected to have a content of 75.7%. The yield of the intermediate IIC in three steps of this example is 42.9%, and the reaction yield of this example is slightly low. The inventors of the present application speculate that it may be affected by residual trace water in the preparation of the 2-methoxyethanol salt.

### Example 8

### Synthesis of ethyl 2-((2-methoxyethoxy)methyl)-6-(trifluoromethyl) nicotinate (intermediate IIC)

A mixture (prepared according to step 1 of Example 4, 20.42 g, 100 mmol) of ethanol (50 g), ethyl 4-(2-methoxyethoxy)-3-oxobutyrate and ethyl (Z)-3-hydroxy-4-(2-methoxyethoxy)-3-oxobutyrate was added to a 250 mL four-necked round-bottom flask. The four-necked flask was placed in a cold trap at a temperature of 0° C. Stirring started; after the temperature of the reaction liquid was reduced to 5±3° C., a 20% ethanol solution of sodium ethoxide (34.04 g, 100 mmol) was added dropwise using a constant-pressure dropping funnel; and the dropwise addition was completed at about 30 min. After the dropwise addition was completed, the temperature was kept for reaction for 0.5 h. A toluene (90 mL) solution of 4-ethoxy-1,1,1-trifluorobut-3-en-2-one (18.28 g, 109 mmol) was then added dropwise; and the dropwise addition was completed at about 20 min. After the dropwise addition was completed, the temperature was kept for reaction for 1 h. Deionized water (100 g) was added to a 30% hydrochloric acid solution (12.40 g) for uniform mixing. The reaction liquid was directly poured into acid water, and extracted with dichloromethane (80 g) to separate out an organic phase; an aqueous phase was extracted with dichloromethane (20 g); and the organic phases were combined; dichloromethane was recovered under reduced pressure; and a residue was subjected to vacuum distillation at 55° C. to obtain an intermediate IC (32.63 g).

Acetic acid (130 g), ammonium acetate (9.37 g, 122 mmol) and the obtained intermediate IC (32.63 g) were added to a 250 mL four-necked round-bottom flask, heated to 50° C. in an oil bath; and the temperature was kept for reaction for 2 h. The temperature was raised to 65° C.; vacuum distillation was performed at 2 mmHg until no fraction was distilled; and deionized water (100 g) and dichloromethane (100 g) were added to the four-necked flask for extraction to separate out an organic phase. The aqueous phase was extracted with dichloromethane (30 g×2); the organic phases were combined; a solvent was removed under reduced pressure; and a residue was heated to 60° C., and subjected to vacuum distillation at a pressure smaller than or equal to 0.095 Mpa to obtain a yellow and black oily mater of 27.2 g with a content being 89.2%. The yield of the intermediate IIC in two steps of this example is 79.0%.

### Example 9

### Synthesis of ethyl 2-((2-methoxyethoxy)methyl)-6-(trifluoromethyl) nicotinate (intermediate IIC)

A mixture (prepared according to step 1 of Example 4, 10.21 g, 50 mmol) of toluene (51.05 g), ethyl 4-(2-methoxyethoxy)-3-oxobutyrate and ethyl (Z)-3-hydroxy-4-(2-methoxyethoxy)-3-oxobutyrate was added to a 250 mL four-necked round-bottom flask for stirring at a constant temperature of 25° C.; and a sodium ethoxide solid (4.08 g, 60 mmol) was added for reaction for 0.5 h. The reaction liquid was cooled to 5° C.±3° C.; a mixed solution of 4-ethoxy-1,1,1-trifluorobut-3-en-2-one (9.14 g, 54 mmol) and toluene (10.21 g) was added dropwise; and the dropwise addition was completed at about 20 min. After the dropwise addition was completed, the temperature was kept for reaction for 1 h. The reaction was detected by a TLC spot plate; and after the raw materials reacted completely, the reaction was stopped. Deionized water (51.05 g) was added to a 30% hydrochloric acid solution (7.9 g), and acid water was directly poured into the reaction liquid for stirring for 10 min. Liquid separation was performed to obtain a toluene solution of IC.

The toluene solution of the intermediate IC was transferred into a 250 mL four-necked round-bottom flask, and acetic acid (3.00 g) and ammonium acetate (4.68 g, 61 mmol) were added. The four-necked flask was placed in an oil bath pot, and heated to 50° C.; and the temperature was kept for reaction for 2 h. The reaction was detected by a TLC spot plate; and after the raw materials reacted completely, the reaction was stopped. The reaction liquid was transferred into a separatory funnel, for standing and liquid separation. An upper organic phase was taken, and subjected to vacuum distillation with a water pump until no fraction was distilled; and then distillation was stopped. 14.2 g of a ring-closure product was obtained with a content being 88.6%. The yield of the intermediate IIC in this example is 81.9%.

### Example 10

### Synthesis of ethyl 2-((2-methoxyethoxy)methyl)-6-(trifluoromethyl) nicotinate (intermediate IIC)

A mixture (prepared according to step 1 of Example 4, 10.21 g, 50 mmol) of toluene (51.05 g), ethyl 4-(2-methoxyethoxy)-3-oxobutyrate and ethyl (Z)-3-hydroxy-4-(2-methoxyethoxy)-3-oxobutyrate was added to a 250 mL four-necked round-bottom flask for stirring at a constant temperature of 25° C.; and sodium carbonate (6.36 g, 60 mmol) was added for reaction for 0.5 h. The reaction liquid was cooled to 5° C.±3° C.; a mixed solution of 4-ethoxy-1,1,1-trifluorobut-3-en-2-one (9.14 g, 54 mmol) and toluene (10.21 g) was added dropwise; and the dropwise addition was completed at about 20 min. After the dropwise addition was completed, the temperature was kept for reaction for 1 h.

The reaction was detected by a TLC spot plate; and after the raw materials reacted completely, the reaction was stopped. Deionized water (51.05 g) was added to a 30% hydrochloric acid solution (7.9 g), and the acid water was directly poured into the reaction liquid for stirring for 10 min. Liquid separation was performed to obtain a toluene solution of IC.

The toluene solution of the IC was transferred into a 250 mL four-necked round-bottom flask, and acetic acid (3.00 g) and ammonium acetate (4.68 g, 61 mmol) were added. The four-necked flask was placed in an oil bath pot, and heated to 50° C.; and the temperature was kept for reaction for 2 h. The reaction was detected by a TLC spot plate; and after the raw materials reacted completely, the reaction was stopped. The reaction liquid was transferred into a separatory funnel, for standing and liquid separation. An upper organic phase was taken, and subjected to vacuum distillation with a water pump until no fraction was distilled; and then distillation was stopped. 13.9 g of a ring-closure product was obtained with a content being 90.7%. The yield of the intermediate IIC in this example is 82.1%.

### Example 11 (One-Pot Method)

### Synthesis of ethyl 2-((2-methoxyethoxy)methyl)-6-(trifluoromethyl) nicotinate (intermediate IIC)

Sodium ethoxide (8.46 g, 124.3 mmol) was added to 2-methoxyethanol (28.4 g, 373.2 mmol) for stirring and heating; a resultant was heated in an oil bath to 130° C., and subjected to vacuum distillation to collect 12 g of a fraction; the temperature was descended to below 100° C.; and toluene (51.1 g) was added for stirring and dispersing to obtain a toluene solution of a sodium salt of 2-methoxyethanol.

The temperature was controlled at about 30° C., and ethyl 4-chloroacetoacetate (8.89 g, 54 mmol) was added to the toluene solution of the sodium salt of 2-methoxyethanol dropwise; after the addition was completed, the temperature was kept at 40° C.; stirring was performed for a reaction for 6 h; and TLC (PE:EA=6:1) tracking detection was performed until the raw materials reacted completely.

The system was cooled to make the reaction liquid cooled to 5° C.±3° C.; a mixed solution of 4-ethoxy-1,1,1-trifluorobut-3-en-2-one (9.14 g, 54 mmol) and toluene (10.21 g) was added dropwise; and the dropwise addition was completed at about 20 min. After the dropwise addition was completed, the temperature was kept for reaction for 1 h. The reaction was detected by a TLC spot plate; and after the raw materials reacted completely, acetic acid (4.20 g) and ammonium acetate (4.68 g, 61 mmol) were added. The four-necked flask was placed in an oil bath pot, and heated to 50° C.; and the temperature was kept for reaction for 2 h. The reaction was detected by a TLC spot plate; and after the raw materials reacted completely, the reaction was stopped. The reaction liquid was transferred into a separatory funnel, for standing and liquid separation. An upper organic phase was taken, and subjected to vacuum distillation with a water pump until no fraction was distilled; and then the distillation was stopped. 15.1 g of a ring-closure product was obtained with a content being 88.7%. The yield of the intermediate IIC in this example is 80.7%.

### Example 12 (One-Pot Method)

### Synthesis of ethyl 2-((2-methoxyethoxy)methyl)-6-(trifluoromethyl) nicotinate (intermediate IIC)

Sodium methoxide (6.71 g, 124.3 mmol) was added to 2-methoxyethanol (28.4 g, 373.2 mmol) for stirring and heating; a resultant was heated in an oil bath to 130° C., and subjected to vacuum distillation to collect 6.6 g of a fraction; the temperature was descended to below 100° C.; and toluene (51.1 g) was added for stirring and dispersing to obtain a toluene solution of a sodium salt of 2-methoxyethanol.

The temperature was controlled at about 30° C., and ethyl 4-chloroacetoacetate (8.89 g, 54 mmol) was added to the toluene solution of the sodium salt of 2-methoxyethanol dropwise; after the addition was completed, the temperature was kept at 40° C.; stirring was performed for a reaction for 6 h; and TLC (PE:EA=6:1) tracking detection was performed until the raw materials reacted completely.

The system was cooled to make the reaction liquid cooled to 5° C.±3° C.; a mixed solution of 4-ethoxy-1,1,1-trifluorobut-3-en-2-one (9.14 g, 54 mmol) and toluene (10.21 g) was added dropwise; and the dropwise addition was completed at about 20 min. After the dropwise addition was completed, the temperature was kept for reaction for 1 h. The reaction was detected by a TLC spot plate; and after the raw materials reacted completely, acetic acid (4.20 g) and ammonium acetate (4.68 g, 61 mmol) were added. The four-necked flask was placed in an oil bath pot, and heated to 50° C.; and the temperature was kept for reaction for 2 h. The reaction was detected by a TLC spot plate; and after the raw materials reacted completely, the reaction was stopped. The reaction liquid was transferred into a separatory funnel, for standing and liquid separation. An upper organic phase was taken, and subjected to vacuum distillation with a water pump until no fraction was distilled; and then the distillation was stopped. 14.8 g of a ring-closure product was obtained with a content being 92.5%. The yield of the intermediate IIC in this example is 82.5%.

### Example 13

### Synthesis of ethyl 4-ethoxy-3-oxobutyrate and ethyl (Z)-4-ethoxy-3-hydroxybut-2-enoate (impurity compound E, a pair of keto and enol isomers)

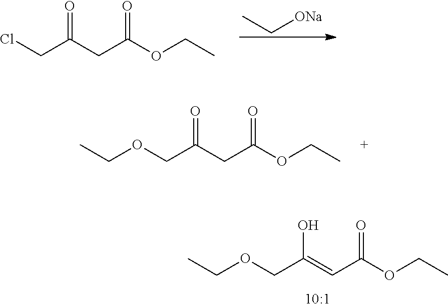

Ethanol (33.6 g) and ethyl 4-chloroacetoacetate (16.8 g, 100 mmol) were added to a 250 mL four-necked flask, and a 20% ethanol solution (68.01 g, 200 mmol) of sodium ethoxide was added while stirring. The temperature was raised to 50° C., and kept for 2 h; GC detection was performed until the content of the raw materials is smaller than or equal to 1%; and then the reaction was stopped. The reaction liquid was poured into a prepared hydrochloric acid solution (150 mL) with a pH value being 2-3. The resultant was exacted with dichloromethane (150 mL); an aqueous phase was extracted with dichloromethane (100 mL×2) for two times; and organic phases were combined. The organic phase was washed with saturated salt water once. The organic phase was separated out, dried with anhydrous magnesium sulfate, and concentrated to obtain a crude product which was a light yellow liquid. The crude product was purified by column chromatography to obtain an analytical sample, which had a content larger than or equal to 97%, detected by GC. The structure was confirmed by GC-MS and NMR standards. NMR proves that a ratio of a keto structure to an enol structure is 10:1. The preparation of the impurity compound E is to characterize the impurity compound E in the reaction route of the present application.

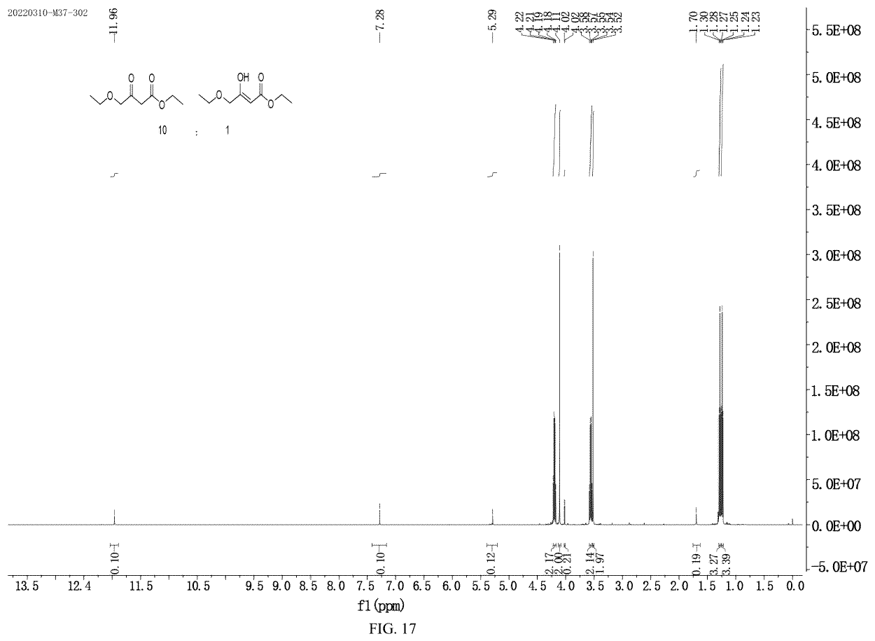

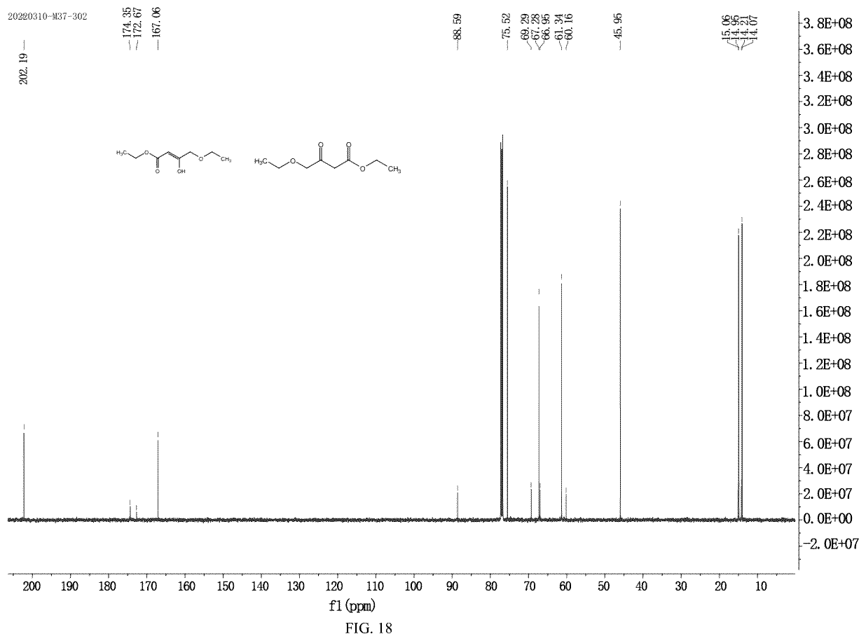

Nuclear magnetic H and C spectral analysises on the impurity compound E are shown FIGS. 17 and 18.

GC-MS: M=174

¹H NMR (CDCl₃, 500 MHz), δ (ppm): (Ketone) 4.20 (q, 2H, J=5.0 Hz), 4.11 (s, 2H), 3.57 (q, 2H, J=5.0 Hz), 3.52 (s, 2H), 3.63 (t, 2H, J=5.0 Hz), 1.28 (t, 3H, J=5.0 Hz), 1.24 (t, 3H, J=5.0 Hz)

¹³C NMR (CDCl₃, 150 MHz), δ (ppm): (Ketone) 202.19, 167.06, 75.52, 67.28, 61.34, 45.95, 14.95, 14.07; (Enol) 174.35, 172.67, 88.59, 69.29, 66.95, 60.16, 15.06, 14.21

### Example 14

### Synthesis of ethyl 2,5-dihydroxycyclohexane-1,4-diene-1,4-dicarboxylate (impurity compound C)

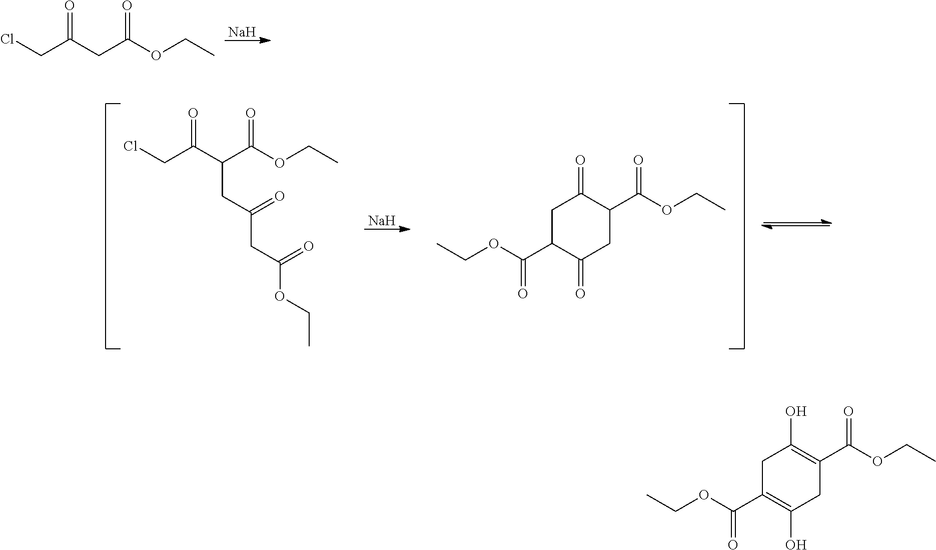

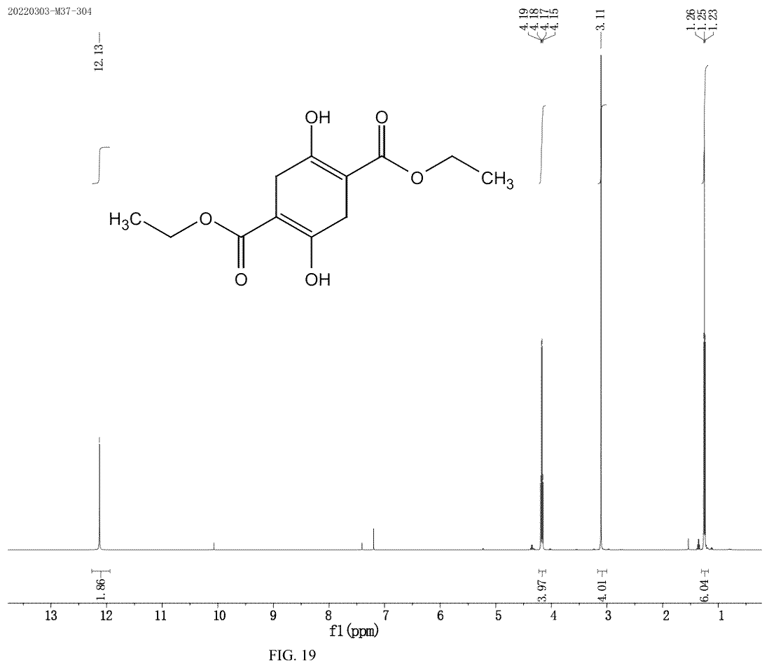

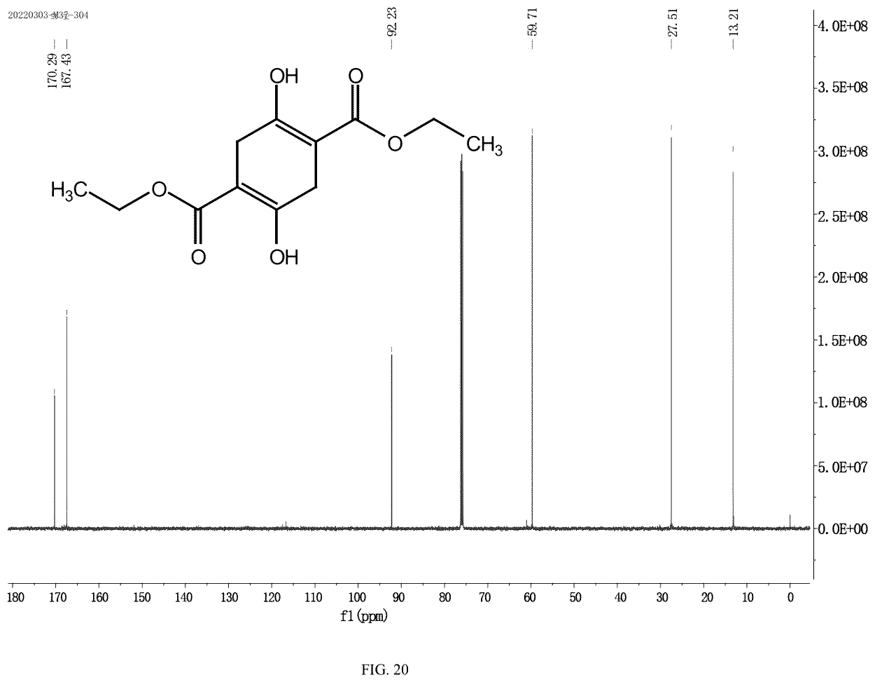

Tetrahydrofuran (40.0 g) was added to a 250 mL four-necked flask, and a temperature was controlled to below 15° C.; sodium hydride (2.4 g, 60%, 60 mmol) was added in batches while stirring for 10 min; then the resultant was cooled to 5° C.-10° C.; an ethyl 4-chloroacetoacetate (10 g, dissolved in 30 mL of tetrahydrofuran, 61 mmol) solution was slowly added dropwise; the four-necked flask was put in an ice water bath for cooling, followed by constant production of bubbles. After the dropwise addition was completed, the reaction liquid was light yellowish brown. The temperature was gradually raised to 25° C.-30° C., and the reaction liquid gradually became brown yellow and clear. A solvent was removed by vacuum rotary evaporation; water (150 mL) was added; dichloromethane (150 mL) was added while stirring; and a pH value was regulated to 2-3. Extraction and liquid separation were performed; an aqueous phase was extracted with dichloromethane (100 mL×2) for two times; and organic phases were combined. The organic phase was washed with saturated salt water once. The organic phase was separated out, dried with anhydrous magnesium sulfate, and concentrated to obtain a crude product. The crude product was subjected to column chromatography to obtain a product of 2.72 g with a yield being 35.4%. The product was purified by recrystallization and purification to obtain an analytical sample, which was a light yellow crystal. Nuclear magnetic H and C spectral analysises on the impurity compound C are shown FIGS. 19 and 20.

LC-MS: M−1=255, M+1=257

¹H NMR (CDCl₃, 500 MHz), δ (ppm): 12.13 (s, 2H), 4.18 (t, 4H, J=5.0 Hz), 3.11 (s, 4H), 1.25 (t, 6H, J=5.0 Hz)

¹³C NMR (CDCl₃, 150 MHz), δ (ppm): 170.29, 167.43, 92.23, 59.71, 27.51, 13.21.

Finally, it should be stated that: the above examples are only for illustrating the technical solutions of the present application rather than to limit the technical solutions of the present application. While the present application is described in detail in reference with the foregoing examples, a person of ordinary skill in the art should understand that the technical solutions recited in the foregoing examples may still be modified, or part of the technical features therein are substituted with equivalents; however, these modifications or substitutions do not make the essence of the corresponding technical solutions depart from the spirit and the scope of the technical solutions of various examples of the present application.

### INDUSTRIAL APPLICABILITY

The present application provides a compound, a preparation method therefor and an application of the compound in the preparation of a bicyclopyrone intermediate, where a bicyclopyrone intermediate II may be prepared at a high yield through an intermediate I (having the structural formula as shown in the formula Ia and/or the formula Ib), or a pharmaceutically acceptable salt thereof, a solvate thereof and a tautomer of Ib (the compound as shown in the formula Ic). In the present application, a one-pot method of producing the intermediate I under the action of a base and then performing a ring-closure reaction to produce a bicyclopyrone intermediate (II), can reduce side reactions, and can further increase the yield.

1-20. (canceled)

21. A compound, having a structure as shown in the formula Ia or the formula Ib, or a pharmaceutically acceptable salt thereof, or a solvate thereof, or a tautomer Ic of Ib, 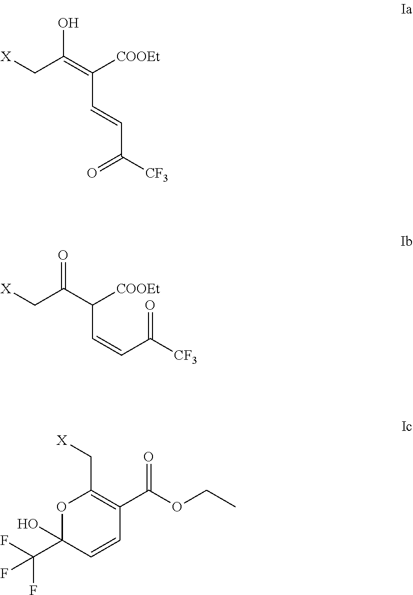
wherein X is —O—R¹—O—R², —H, —Cl or —Br; and if X is —O—R¹—O—R², R¹ is selected from C₁-C₄ alkylene groups, and R² is selected from C₁-C₄ alkyl groups.

22. The compound according to claim 21, wherein X is —H or —Cl;
or X is —O—R¹—O—R², R¹ is selected from C₁-C₄ alkylene groups, and R² is selected from C₁-C₂ alkyl groups; or R¹ is selected from C₁-C₃ alkylene groups, and R² is selected from C₁-C₂ alkyl groups;
Optionally, if X is —O—R¹—O—R², R¹ is selected from C₂-C₃ alkylene groups, and R² is selected from C₁-C₂ alkyl groups; and if X is —O(CH₂)₂OCH₃, the compound has a structure as shown in the formula Ib-3, or a tautomer as shown in the formula Ic-3,

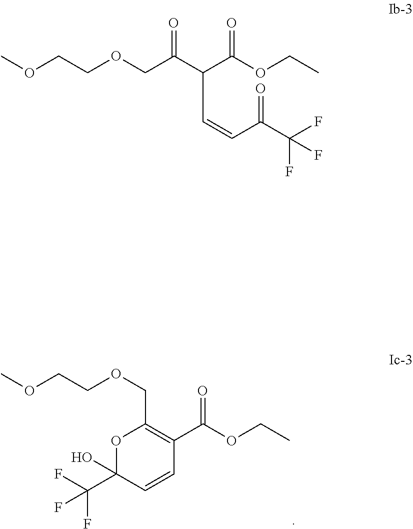 [SMILES: CCOC(=O)C(/C=C\C(=O)C(F)(F)F)C(=O)COCCOC ; CCOC(=O)C1=C(COCCOC)OC(O)(C(F)(F)F)C=C1]

23. A preparation method for the compound according to claim 21, comprising the following step:
in the presence of a base, making a compound as shown in the formula III and/or an enol tautomer thereof subjected to a substitution reaction with a compound as shown in the formula IV, to obtain a compound as shown in the formula Ia and/or Ib and/or Ic, 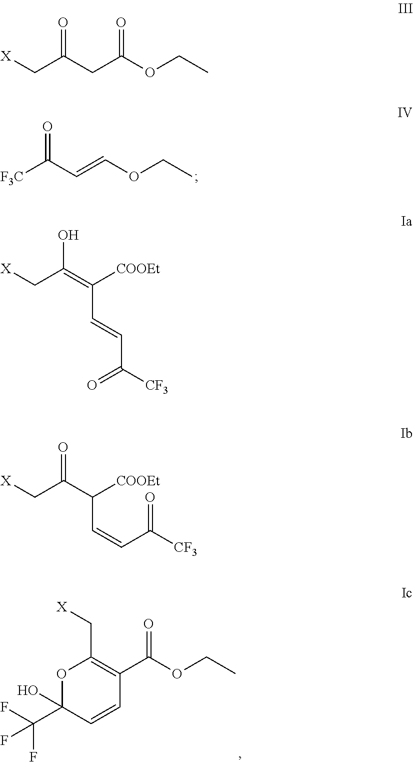
wherein X is —O—R¹—O—R, —H, —Cl or —Br; and if X is —O—R¹—O—R², R¹ is selected from C₁-C₄ alkylene groups, and R² is selected from C₁-C₄ alkyl groups; and
In the formulas Ia, Ib, Ic, II and III, X is the same.

24. The preparation method according to claim 23, wherein in the substitution reaction, the base is one or more selected from the group consisting of an organic base, an inorganic base, sodium hydride or metal sodium, wherein the organic base comprises one or more of sodium alkoxide and potassium alkoxide; the organic base comprises one or more of sodium methoxide, sodium ethoxide, potassium tert-butoxide, potassium methoxide, potassium ethoxide, sodium hexamethyldisilazane and lithium hexamethyldisilazane; the inorganic base comprises one or more of sodium hydroxide, potassium hydroxide, sodium carbonate, potassium carbonate, sodium phosphate, potassium phosphate and sodium amide; optionally, the base is one or more selected from the group consisting of sodium methoxide, sodium ethoxide, sodium hydroxide and sodium carbonate; and/or,
the substitution reaction is performed in an organic solvent, and the organic solvent comprises one or more of organic alcohol, toluene, tetrahydrofuran, dimethyl sulfoxide, N, N-dimethylformamide, and 1, 4-dioxane; optionally, the organic solvent comprises one or more of methanol, ethanol and toluene; and/or,
a reaction temperature for the substitution reaction is −15° C. to 30° C.; optionally, the reaction temperature for the substitution reaction is 0° C.-25° C. or 0° C.-10° C.; and/or,
a molar ratio of the compound as shown in the formula IV, the compound as shown in the formula III and/or an enol tautomer thereof and the base is 1:0.8-1.5:0.05-1.5, optionally, 1:0.8-1.2:0.5-1.3, optionally, 1:0.9-1.1:1-1.3, or 1:1: 1-1.3, or 1:1: 1-1.2.

25. A preparation method for a bicyclopyrone intermediate, comprising the following steps:
1) in the presence of a base, making a compound as shown in the formula III and/or an enol tautomer thereof subjected to a substitution reaction with a compound as shown in the formula IV, 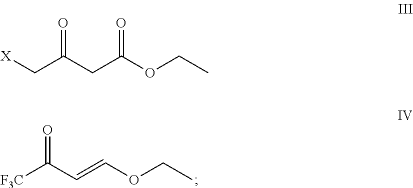
2) in the presence of an ammonium salt and/or ammonia, making a product of the substitution reaction in step 1) subjected to a ring-closure reaction to obtain a compound as shown in the formula II, wherein in the formula II and the formula III, X is the same,

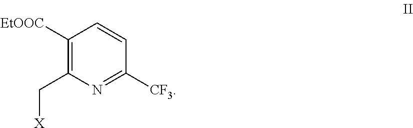

26. The preparation method according to claim 25, wherein the condition for the ring-closure reaction in step 2) comprises: a temperature is 45° C.-80° C., optionally, 45° C.-60° C.;
and/or,
in the ring-closure reaction in step 2), the ammonium salt comprises one or more of ammonium chloride, ammonium carbonate, ammonium bicarbonate, ammonium nitrate, ammonium sulfate, ammonium phosphate, and ammonium acetate, and the ammonia exists in a form of ammonia gas and/or ammonia water, optionally, the ammonium salt comprises ammonium acetate; and/or,
in the ring-closure reaction in step 2), a molar ratio of the product of the substitution reaction in step 1) to the ammonium salt and/or the ammonia is 1: 1-5, optionally, 1: 1-2.5, optionally, 1:1.2-1.5.

27. The preparation method according to claim 25, wherein if X is Cl, the product of the substitution reaction further comprises at least one of impurity compounds as shown in the following formulas:

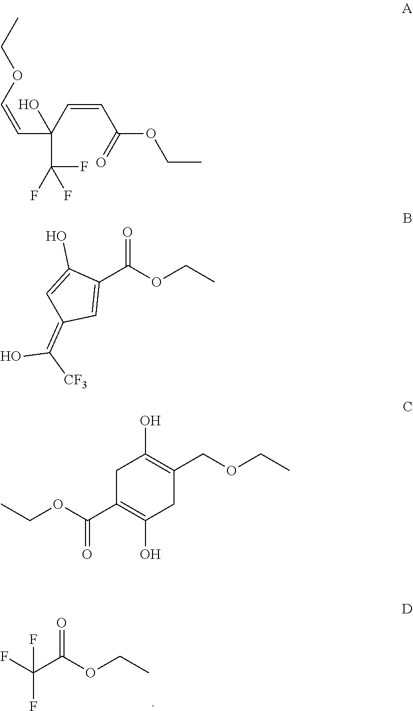

28. The preparation method according to claim 25, wherein if X is —O(CH₂)₂OCH₃, the product of the substitution reaction further comprises at least one of an impurity compound C, an compound D and an compound E,

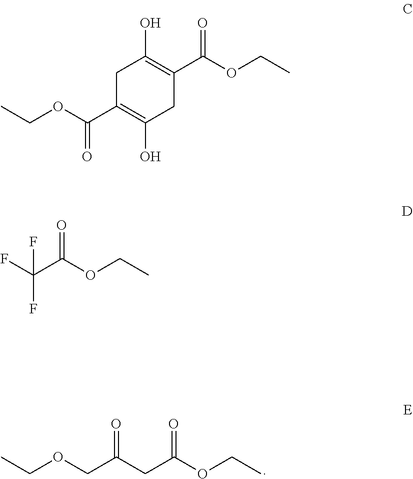

29. The preparation method according to claim 26, wherein if X is —O(CH₂)₂OCH₃, the product of the substitution reaction further comprises at least one of an impurity compound C, an compound D and an compound E,

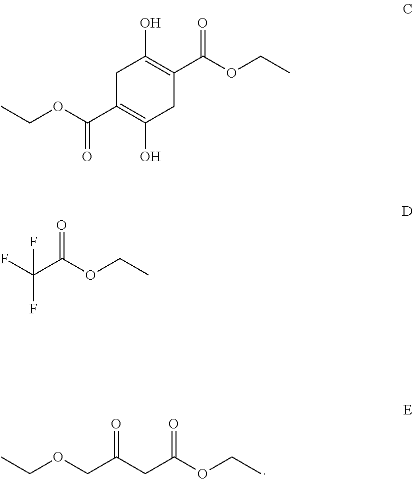

30. A composition, comprising the compound according to claim 21;
if X is H, the composition comprises a compound having the structural formula as shown in the formula Ia, the formula Ib and/or the formula Ic;
if X is Cl, the composition further comprises at least one of an impurity compound A, an compound B, an compound C and an compound D; and
if X is —O(CH₂)₂OCH₃, the composition further comprises at least one of an impurity compound C, an compound D and an compound E.

31. A composition, comprising a reaction material prepared by the method according to claim 23;
if X is H, the composition comprises a compound having the structural formula as shown in the formula Ia, the formula Ib and/or the formula Ic;
if X is Cl, the composition further comprises at least one of an impurity compound A, an compound B, an compound C and an compound D; and
if X is —O(CH₂)₂OCH₃, the composition further comprises at least one of an impurity compound C, an compound D and an compound E.

32. A composition, comprising a reaction material prepared by the method according to claim 25;
if X is H, the composition comprises a compound having the structural formula as shown in the formula Ia, the formula Ib and/or the formula Ic;
if X is Cl, the composition further comprises at least one of an impurity compound A, an compound B, an compound C and an compound D; and
if X is —O(CH₂)₂OCH₃, the composition further comprises at least one of an impurity compound C, an compound D and an compound E.

33. A method for the preparation of bicyclopyrone from the compound according to claim 21.

34. A method for the preparation of bicyclopyrone from the product prepared by the method according to claim 23.

35. A method for the preparation of bicyclopyrone from the product prepared by the method according to claim 25.

36. A preparation method for a bicyclopyrone intermediate IIC, comprising the following steps:
(1) making a material containing a salt of 2-methoxyethanol reacted with ethyl 4-chloroacetoacetate, to obtain a reaction material containing the formula III-a and/or III-b; 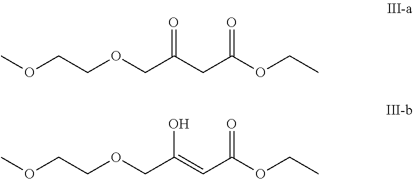
(2) making the reaction material in step (1) subjected to a substitution reaction with a compound as shown in the formula IV under the action of a base, to obtain a reaction material; 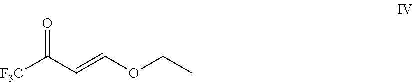
and
(3) obtaining a compound as shown in the formula IIC by adding an ammonium salt and/or ammonia to the reaction material in step (2) to make the reaction material in step (2) subjected to a ring-closure reaction,

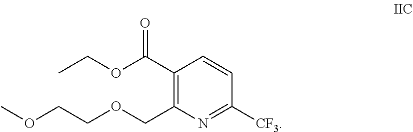 [SMILES: CCOC(=O)c1ccc(C(F)(F)F)nc1COCCOC]

37. The preparation method according to claim 36, wherein in step (2), a reaction temperature for the substitution reaction is −15° C. to 30° C. or 0° C.-10° C.

38. The preparation method according to claim 36, wherein in step (3), a reaction temperature for the ring-closure reaction is 0° C.-80° C., optionally, 30° C.-80° C., optionally, 45° C.-60° C.; and/or
in step (3), the ammonium salt comprises one or more of ammonium chloride, ammonium carbonate, ammonium bicarbonate, ammonium nitrate, ammonium sulfate, ammonium phosphate, and ammonium acetate, and the ammonia exists in a form of ammonia gas and/or ammonia water, optionally, the ammonium salt comprises ammonium acetate; and/or
in step (3), a molar ratio of a compound as shown in the formula Ib-3 to the ammonium salt and/or the ammonia is 1: 1-5, optionally, 1: 1-2.5, optionally, 1:1.2-1.5.

39. The preparation method according to claim 36, wherein the reaction material in step (2) further comprises at least one of an impurity compound C, an impurity compound D and an impurity compound E,

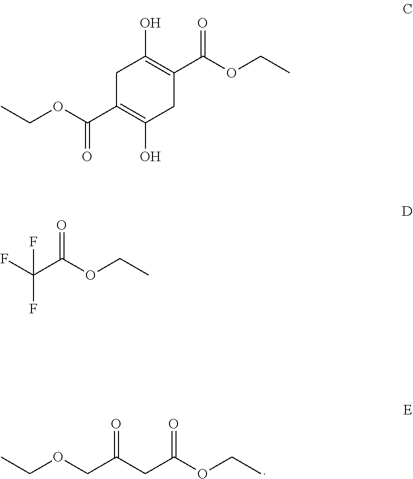

40. The preparation method according to claim 36, wherein the reaction material in step (1) is directly used for the reaction in step (2); and/or,
the reaction material in step (2) is directly used for the reaction in step (3).

41. The preparation method according to claim 36, wherein in step (1), the material containing a salt of 2-methoxyethanol is a reaction material obtained by a reaction of 2-methoxyethanol under the action of a base, wherein a molar ratio of an addition amount of the base and the compound as shown in the formula IV is 1-3:1, optionally, 2-2.5:1, optionally, 2-2.3:1;
optionally, the base is one or more selected from the group consisting of an organic base, an inorganic base, sodium hydride or metal sodium, the organic base comprises one or more of sodium alkoxide and potassium alkoxide; the organic base comprises one or more of sodium methoxide, sodium ethoxide, potassium tert-butoxide, potassium methoxide and potassium ethoxide; and the inorganic base comprises one or more of sodium hydroxide, potassium hydroxide, and sodium amide.

42. A preparation method for a bicyclopyrone intermediate, comprising the following steps:
S1: performing a reaction on 2-methoxyethanol under the action of a base, to obtain a material containing a salt of 2-methoxyethanol; and
S2: making the material containing a salt of 2-methoxyethanol reacted with ethyl 4-chloroacetoacetate, to obtain a reaction material containing the formula III-a and/or III-b,

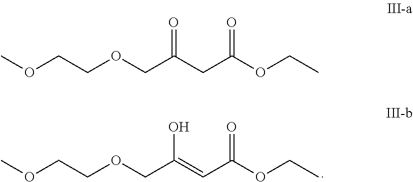

43. The preparation method according to claim 42, wherein in step S1, the base is one or more selected from the group consisting of an organic base, an inorganic base, sodium hydride or metal sodium, the organic base comprises one or more of sodium alkoxide and potassium alkoxide; the organic base comprises one or more of sodium methoxide, sodium ethoxide, potassium tert-butoxide, potassium methoxide and potassium ethoxide; and the inorganic base comprises one or more of sodium hydroxide, potassium hydroxide, and sodium amide.

44. The method according to claim 41, wherein in step (3), a reaction temperature for 2-methoxyethanol and a base is 40° C.-180° C., optionally, 80° C.-150° C., optionally, 80° C.-130° C.; and/or,
the base is one or more selected from the group consisting of sodium ethoxide, sodium methoxide, potassium methoxide and potassium ethoxide.

45. The method according to claim 42, wherein in step (3), a reaction temperature for 2-methoxyethanol and a base is 40° C.-180° C., optionally, 80° C.-150° C., optionally, 80° C.-130° C.; and/or,
the base is one or more selected from the group consisting of sodium ethoxide, sodium methoxide, potassium methoxide and potassium ethoxide.

46. The method according to claim 36, wherein in step (1) or step S2, a molar ratio of ethyl 4-chloroacetoacetate to the salt of 2-methoxyethanol is 1:1.8-2.5 or 1: 2-2.3; and/or, in the reaction material in step (1) or step S2, the compounds as shown in the formula III-a and the formula III-b are a pair of tautomers.

47. The method according to claim 42, wherein in step (1) or step S2, a molar ratio of ethyl 4-chloroacetoacetate to the salt of 2-methoxyethanol is 1:1.8-2.5 or 1: 2-2.3; and/or, in the reaction material in step (1) or step S2, the compounds as shown in the formula III-a and the formula III-b are a pair of tautomers.
